(19) United States
(12) Patent Application Publication
(10) Pub. No.: US 2019/0075794 A1
(43) Pub. Date: Mar. 14, 2019

(54) BIODEGRADABLE ANTIMICROBIAL COMPOSITIONS AND USES THEREOF TO COMBAT MICROORGANISMS

(71) Applicant: IPABC LTD, Daventry, Northamptonshire (GB)

(72) Inventors: ULRICH W. SCHWARZ, RHEINFELD (DE); GRACY SAILO-LEBEAU, LEICESTERSHIRE (GB)

(21) Appl. No.: 15/743,089

(22) PCT Filed: Jul. 8, 2016
(86) PCT No.: PCT/EP2016/066368

§ 371 Date: Jan. 9, 2018

(30) Foreign Application Priority Data
Jul. 10, 2015 (GB) 1512135.3

(51) Int. Cl. A01N 43/16; A01N 37/36

(52) U.S. Cl. CPC A01N 43/16; A61K 31/7004; A01N 37/36

(57) ABSTRACT

The invention provides a method for combating contamination of a site with a microorganism, said method comprising contacting the site and/or the microorganism with an antimicrobial aqueous composition comprising a decylglucoside at a concentration of about 2 mM to about 40 mM and at least one C₁ to C₈ carboxylic acid, or a water-soluble salt thereof, at a concentration of about 1 μM to about 100 mM. The invention further provides such compositions, concentrated forms thereof, dry solid forms thereof and methods and kits for preparing such compositions

The present invention relates to biodegradable antimicrobial compositions and uses thereof to combat microorganisms. More specifically there is provided a synergistic combination of a specific akylglycoside and low molecular weight carboxylic acids which, when combined in an aqueous formulation, exerts antimicrobial effects at concentrations at which no such activity is observed for the individual components. More specifically said formulations are aqueous compositions which comprise a decylglucoside at a concentration of as little as about 2 mM and at least one C₁ to C₈ carboxylic acid, or a water-soluble salt thereof, at a concentration of as little as about 1 μM. The invention therefore provides methods for combating the contamination of a site with a microorganism, particularly large scale industrial, agricultural, institutional and commercial sites, which comprise applying such compositions to such sites. As shown in the Examples, these concentrations of the components of the synergistic combination of the invention are the minimum concentrations having practical antimicrobial effects. Advantageously, the aqueous composition may be provided in a concentrated form which may be diluted with an aqueous diluent to closer to the minimum concentrations at or near the site of application prior to use, thereby minimising the cost and environmental impact of the composition itself and all parts of the production and supply chain. The site to be treated with the compositions of the invention may be in any context, and as such both medical and non-medical uses are provided. Also provided are dry solid, e.g. powder, compositions comprising amounts of a decylglucoside and at least one C₁ to C₈ carboxylic acid, or a water-soluble salt thereof, that following contact with a sufficient amount of an aqueous liquid provides the ready to use antimicrobial aqueous compositions of the invention.

There is a continuing need to develop antimicrobial compositions that have the least impact on the environment possible. Such compositions must be highly effective so as to minimise the amount of antimicrobial compound (and any diluent or excipients) applied. Even if the diluent used is water, there is now pressure to minimise the use of this natural resource. The antimicrobial compounds (and other ingredients) should also be biodegradable, ideally readily so, and non-hazardous to higher organisms. Moreover, the manufacture of such compounds should involve natural compounds as source materials. From a cost perspective the more effective the composition the less must be used, thereby saving on materials costs and manufacturing and transport costs. The provision of concentrated forms, which may be diluted with an aqueous diluent to form a ready to use composition at or near the site of application prior to use, may further minimise the cost and environmental impact of the composition itself and all parts of the production and supply chain. Similarly ready mixed solid, e.g. powder, forms of the components of the compositions of the invention which may be dissolved in an aqueous diluent at or near the site of application prior to use in order to form a ready to use composition will also be a cost and environmentally effective option.

Antimicrobial compositions comprising alkyl glycosides and bactericidally active carboxylic acids have been proposed in U.S. Pat. No. 4,920,100 on the basis of the apparent potentiation of the antibacterial effects of the carboxylic acid by the alkyl glycoside.

In contrast, it has now been surprisingly found that the combination of a decylglucoside at concentrations at or just above the critical micelle concentration (CMC) of the decylglucoside (about 1.8 to about 2 mM or about 300 ppm) with extremely low concentrations of a low molecular weight carboxylic acid, e.g. lactic acid and/or citric acid results in a highly effective antimicrobial composition. Such concentrations of low molecular weight carboxylic acid are orders of magnitude lower than those proposed for use or used in U.S. Pat. No. 4,920,100.

Thus in a first aspect the invention provides a method for combating contamination of a site with a microorganism, said method comprising contacting the site and/or the microorganism with an antimicrobial aqueous composition comprising:
(i) a decylglucoside at a concentration of about 2 mM to about 40 mM, and
(ii) at least one C₁ to C₈ carboxylic acid, or a water-soluble salt thereof, at a concentration of about 1 μM to about 100 mM.

The decylglucosides are a class of alkyl glycoside containing monomeric glucose as the glycone group and a 10 carbon saturated alkyl chain linked to the glucose residue via a glycosidic bond, e.g. an S-, N-, C-, or O-glycosidic bond. Preferably the decylcoside is decyl β-D-glucopyranoside.

For convenience, references herein to the at least one C₁ to C₈ carboxylic acid include water-soluble salts thereof unless context dictates otherwise.

The at least one C₁ to C₈ carboxylic acid may be a C₁ to C₆, C₃ to C₈ , or C₃ to C₆ carboxylic acid. The at least one C₁ to C₈ carboxylic acid may be an aliphatic or an aromatic carboxylic acid containing one or more carboxyl groups. Straight-chain or branched, saturated or unsaturated, unsubstituted or mono- or disubstituted acids may be used. The at least one C₁ to C₈ carboxylic acid may preferably monocarboxylic or dicarboxylic. Suitable substituents are SR¹ (wherein R¹═H or C₁₋₃ alkyl, e.g. —CH₃), NR₂ (wherein R₂ is independently H or C₁₋₃ alkyl, e.g. —CH₃), Cl, Br, NO₂ and OH. Preferably the at least one C₁ to C₈ carboxylic acid is an alpha hydroxy acid (AHA), e.g. lactic acid, citric acid, glycolic acid, malic acid, mandelic acid and tartaric acid. The at least one C₁ to C₈ carboxylic acid may for example be, e.g. lactic acid, citric acid, glycolic acid, malic acid, mandelic acid, tartaric acid, butyric acid, succinic acid, sorbic acid, isocitric acid, aconitic acid, propane-1,2,3-tricarboxylic acid, propionic acid, valeric acid, benzoic acid, oxalic acid or salicylic acid. Lactic acid and citric acid are preferred. If isomeric forms are available, any isomeric form or mixture thereof, e.g. racemic, may be used. In certain embodiments the L(+) isomer only is used, e.g. L(+) lactic acid.

Water-soluble salts of the recited carboxylic acids are primarily alkali or alkali earth metal salts, of which the sodium, potassium and calcium salts are preferably used. Preferably the water-soluble salts are non-hazardous to humans and higher organisms, especially in the context of the medical uses of the invention.

By “water soluble” it is meant that the salt can be dissolved to the required concentration in the antimicrobial aqueous composition. Conveniently water soluble salts of the at least one C₁ to C₈ carboxylic acid is a salt for which less than 1000 parts pure water are required to solubilise 1 part of the salt, e.g. less than 500, 250, 100, 50, 40, 30, 20, 10, 9, 8, 7, 6, 5, 4, 3, 2 or 1 parts pure water are required to solubilise 1 part of the salt.

Any number of C₁ to C₈ carboxylic acids or water-soluble salts thereof may be used in the compositions of the invention, e.g. 1, 2, 3, 4, 5, 7, 10 or 15 or more, preferably less than 5, 4, 3, e.g. 1 or 2. A combination of lactic acid or water-soluble salt thereof and citric acid or water-soluble salt thereof may be advantageous. In embodiments in which a plurality is used it is the combined (or total) concentration of said acids and/or salts which is considered as the concentration of the “at least one C₁ to C₈ carboxylic acid or water-soluble salt thereof”

“Combating contamination” includes both preventative and reactionary measures or treatments and therefore covers the prevention as well as the reduction, limitation, inhibition or elimination of contamination. The term may be considered to cover “sanitising”, “disinfecting” or “sterilising” measures or treatments. To disinfect or to sanitise is to reduce levels of microbes to those considered safe by local public health ordinance over a preselected timeframe, e.g. 24 hrs. To sterilise is to eliminate microbes over a preselected timeframe, e.g. 24 hrs.

By “contamination” it is meant the unwanted presence of a microorganism at a particular site or location. In abiotic locations this can be considered at its extreme to refer to the presence of any microorganism at the site. Contamination can be considered to cover colonisation of a location by the microorganism, i.e. the establishment of a microorganism at a location and the expansion of the numbers of that microorganism by replication or the recruitment of additional microorganisms, which may be of the same or of a different type.

More particularly the site (or location) and/or microorganism (or colony thereof) will be contacted with an effective amount of the antimicrobial aqueous composition, more particularly an amount of the antimicrobial aqueous composition sufficient to kill or inhibit the growth of the microorganism (or colony thereof). Expressed differently, the site may be considered to be “sanitised”, “disinfected” or “sterilised”. Colonisation or contamination may be prevented therefore.

The site or location is not restricted, although it is typically a surface. The surface is not limited and includes any surface on which a microorganism may occur. The surface may be biotic or abiotic, and inanimate (or abiotic) surfaces include any such surface which may be exposed to microbial contact or contamination. Thus particularly included are large-scale industrial, agricultural, commercial and institutional (e.g. clinical) surfaces, e.g. those on machinery, notably industrial machinery, or medical equipment or any surface exposed to an aquatic environment (e.g. marine equipment, or ships or boats or their parts or components), or any surface exposed to any part of the environment, e.g. pipes or on buildings. Surfaces in domestic settings are also included, e.g. surfaces in kitchens, lavatory, washroom and utility areas.

Such inanimate surfaces exposed to microbial contact or contamination include in particular any part of: food or drink processing, preparation, storage, packaging or dispensing machinery or equipment (in particular meat processing or packaging machinery or equipment and abattoir machinery or equipment, but also fruit and vegetable processing or packaging machinery or equipment); air conditioning apparatus; industrial machinery, e.g. in chemical or biotechnological processing plants; storage tanks; medical or surgical equipment; clinical beds, furniture, floors, doors, door handles, operating theatres, office and IT equipment; cell and tissue culture equipment; and sanitaryware and fittings. Any apparatus or equipment for carrying or transporting or delivering materials is susceptible to microbial contamination. Such surfaces will include particularly pipes (which term is used broadly herein to include any conduit or line). Representative inanimate or abiotic surfaces include, but are not limited to food processing, storage, dispensing, preparation or packaging equipment or surfaces, tanks, conveyors, floors, drains, coolers, freezers, equipment surfaces, walls, valves, belts, pipes, air conditioning conduits, cooling apparatus, food or drink dispensing lines, heat exchangers, boat hulls or any part of a boat's structure that is exposed to water, dental waterlines, oil drilling conduits, contact lenses and storage cases.

Medical or surgical equipment or devices represent a particular class of surface on which microbial contamination may form. This may include any kind of line, including catheters (e.g. central venous and urinary catheters), prosthetic devices e.g., heart valves, artificial joints, false teeth, dental crowns, dental caps and soft tissue implants (e.g. breast, buttock and lip implants). Any kind of implantable (or “in-dwelling”) medical device is included (e.g. stents, intrauterine devices, pacemakers, intubation tubes (e.g. endotracheal or tracheostomy tubes), prostheses or prosthetic devices, lines or catheters). An “in-dwelling” medical device may include a device in which any part of it is contained within the body, i.e. the device may be wholly or partly in-dwelling. Also included is medical equipment operated by medical staff or technicians that does not contact a patient.

The surface can be made of any material. Substantially, non-absorbent surfaces (“hard” surfaces) may respond best, but the invention is not limited thereto. For example the surface may be metal, e.g. aluminium, steel, stainless steel, chrome, titanium, iron, alloys thereof, and the like. The surface can also be plastic, for example, polyolefin (e.g., polyethylene, (Ultra-High Molecular Weight) polyethylene, polypropylene, polystyrene, poly(meth)acrylate, acrylonitrile, butadiene, ABS, acrylonitrile butadiene, etc.), polyester (e.g., polyethylene terephthalate, etc.), and polyamide (e.g., nylon), combinations thereof, and the like. Other examples include acetal copolymer, polyphenylsulfone, polysulfone, polythermide, polycarbonate, polyetheretherketone, polyvinylidene fluoride, poly(methyl methacrylate) and poly(tetrafluoroethylene). The surface can also be silicone, rubber, brick, tile, ceramic, porcelain, wood, vinyl, linoleum, or carpet, combinations thereof, and the like. The surfaces can also be food, for example, beef, poultry, pork, vegetables, fruits, fish, shellfish, combinations thereof, and the like. Foodstuffs, such as those described above, in isolation from an animal or plant body, are considered inanimate for the purposes of the invention.

A biotic or animate surface may include any surface or interface in or on an animal, plant or fungal body. It may accordingly be viewed as a “physiological” or “biological” surface. It may be any internal or external body surface, including of any tissue or organ, which, in the case of an animal body, may include haematological or haematopoietic tissue (e.g. blood). Dead or dying (e.g. necrotic) or damaged (e.g. inflamed or disrupted or broken) tissue is particularly susceptible to microbiological contamination, and such tissue is encompassed by the term “animate” or “biotic”. The surface may be a mucosal or non-mucosal surface.

Representative biotic surfaces include, but are not limited to, any surface in the oral cavity (e.g. teeth, gingiva, gingival crevice, periodontal pocket) the reproductive tract (e.g. cervix, uterus, fallopian tubes), the peritoneum, middle ear, prostate, urinary tract, vascular intima, eye, i.e. ocular tissue (e.g. the conjunctiva lachrymal duct, lachrymal gland, eyelid), corneal tissue, the respiratory tract, lung tissue (e.g. bronchial and alveolar), heart valves, gastrointestinal tract, skin, scalp, nails and the interior of wounds, particularly chronic wounds and surgical wounds, which may be topical or internal wounds. Other surfaces include the exterior of organs, particularly those undergoing transplantation, for example, heart, lungs, kidney, liver, heart valve, pancreas, intestine, corneal tissue, arterial and venous grafts and skin. Skin, wounds and transplant tissue are of note.

As will be clear from the foregoing, the site or location of the contamination or potential contamination is not restricted, e.g. it can be in vitro or in vivo, but particularly in this aspect of the invention it will be an “in vitro” or “ex vivo” site or location (i.e. an inanimate or abiotic site or location, or an animate or biotic site or location that is isolated from (not in or on) a human, animal, plant or fungal body). However, the site or location may be in or on a human or animal subject and in which case a therapeutically effective amount of the antimicrobial composition is administered to the subject.

Thus, it can be seen that in one particular aspect the invention provides a method for inhibiting the viability and/or growth of a microorganism in or on a subject, said method comprising administering an effective amount of the antimicrobial aqueous composition as defined above to a subject in need thereof.

Also provided is an antimicrobial aqueous composition as defined above for use in inhibiting the viability and/or growth of a microorganism in or on a subject.

Alternatively put, this aspect of the invention provides the use of an antimicrobial aqueous composition as defined above, for the manufacture of a medicament for inhibiting the viability and/or growth of a microorganism in or on a subject. This aspect of the invention also provides the use of the above recited components of the antimicrobial aqueous composition as defined above (e.g. a decylglucoside and at least one C₁ to C₈ carboxylic acid, or water soluble salt thereof) for the manufacture of an aqueous antimicrobial composition as defined above for inhibiting the viability and/or growth of a microorganism in or on a subject.

Viewed differently the invention provides an antimicrobial aqueous composition as defined above for use as a therapeutic microbicidal and/or a microbiostatic agent and the use of an antimicrobial aqueous composition as defined above for the manufacture of a microbicidal and/or a microbiostatic medicament having the features of the antimicrobial aqueous composition as defined above. The invention also provides the use of the above recited components of the antimicrobial aqueous composition as defined above (e.g. a decylglucoside and at least one C₁ to C₈ carboxylic acid, or water soluble salt thereof) for the manufacture of a microbicidal and/or a microbiostatic medicament having the features of the antimicrobial aqueous composition as defined above.

These aspects of the invention can also be seen to provide (i) a method for combating, and in particular in the treatment or prevention of, microbial infection in or on an subject said method comprising administering an effective amount of the antimicrobial aqueous composition as defined above to a subject in need thereof; (ii) an antimicrobial aqueous composition as defined above for use in combating, and in particular in the treatment or prevention of, microbial infection in or on an subject; (iii) the use of an antimicrobial aqueous composition as defined above in the manufacture of a medicament for use in combating, and in particular in the treatment or prevention of, microbial infection in or on a subject or (iv) the use of the above recited components of the antimicrobial aqueous composition as defined above (e.g. a decylglucoside and at least one C₁ to C₈ carboxylic acid, or water soluble salt thereof) in the manufacture of a medicament having the features of the antimicrobial aqueous composition as defined above for use in combating, and in particular in the treatment or prevention of, microbial infection in or on a subject. It will be seen in this aspect that the infection may be combated by inhibiting the growth and/or viability of a microorganism in or on a subject.

The term “microorganism” as used herein includes any microbial organism, that is any organism that is microscopic, namely too small to be seen by the naked eye. In particular as used herein the term includes the organisms typically thought of as microorganisms, particularly bacteria, fungi, archaea, algae and protists. The term thus particularly includes organisms that are typically unicellular, but which may have the capability of organising into simple cooperative colonies or structures such as filaments, hyphae or mycelia (but not true tissues) under certain conditions. The microorganism may be prokaryotic or eukaryotic, and may be from any class, genus or species of microorganism. Examples of prokaryotic microorganisms include, but are not limited to, bacteria, including the mycoplasmas, (e.g. Gram-positive, Gram-negative bacteria or Gram test non-responsive bacteria or mycobacteria) and archaeobacteria. Eukaryotic microorganisms include fungi, algae and others that are, or have been, classified in the taxonomic kingdom Protista or regarded as protists, and include, but are not limited to, for example, protozoa, diatoms, protoophyta, and fungus-like molds. The microorganism may be aerobic or anaerobic. The microorganism may be pathogenic or non-pathogenic, or a be spoilage or an indicator microorganism. In particular preferred embodiments the microorganism is pathogenic.

Bacteria or fungi represent preferred classes of microorganism.

The bacteria may be Gram positive or Gram negative bacteria, or indeed Gram-indeterminate bacteria. Within the Gram-negative bacteria the Enterobacteriaceae and the Gram-negative bacteria non-fermenting bacteria are of particular note.

Preferably the bacteria are selected from the following genera: Achromobacter, Acinetobacter, Actinobacillus, Aeromonas, Agrobacterium, Alcaligenes, Alteromonas, Bacteroides, Bartonella, Borrelia, Bordetella, Brucella, Burkholderia, Campylobacter, Cardiobacterium, Chlamydia, Chlamydophila, Chromobacterium, Chyseobacterium, Chryseomonas, Citrobacter, Clostridium, Comamonas, Corynebacterium, Coxiella, Cryptobacterium, Edwardsiella, Eikenella, Enterobacter, Enterococcus, Erwinia, Escherichia, Kingella, Klebsiella, Lactobacillus, Lactococcus, Legionella, Leptospira, Leptotrichia, Leuconostoc, Listeria, Listonella, Mobiluncus, Moraxella, Morganella, Mycobacterium, Mycoplasma, Neisseria, Nocardia, Nocardiopsis, Pantoea, Parachlamydia, Pasteurella, Peptococcus, Peptostreptococcus, Prevotella, Propionibacterium, Proteus, Providencia, Pseudomonas, Ralstonia, Rickettsia, Salmonella, Shewenella, Shigella, Sphingobacterium, Sphingomonas, Staphylococcus, Stenotrophomonas, Streptobacillus, Streptococcus, Streptomyces, Treponem and Yersinia.

Particular mention may be made of bacteria from the genera Pseudomonas, Acinetobacter, Burkholderia, Escherichia, Klebsiella, Streptococcus, Enterococcus, Providencia, Moraxalla, Staphylococcus, e.g. Pseudomonas aeruginosa, Acinetobacter baumannii, Burkholderia spp, E. coli, Klebsiella pneumoniae, Burkholderia cepacia, Burkholderia multivorans, Burkholderia mallei, Burkholderia pseudomallei, Acinetobacter lwoffii, Providencia stuartii, Providencia rettgeri, Providencia alcalifaciens, Klebsiella oxytoca, Pseudomonas anguilliseptica, Pseudomonas oryzihabitans, Pseudomonas plecoglossicida, Pseudomonas luteola, Moraxalla catarrhalis, Enterococcus faecium, Enterococcus faecalis, Streptococcus oralis, Staphylococcus aureus (e.g. MRSA).

The microorganism may also be a, or from a, fungus, including for example fungi that may be, or may have been, classified as protista, e.g. fungi from the genera Candida, Aspergillus, Pneumocystis, Penicillium and Fusarium. Representative fungal species include, but are not limited to, Candida albicans, Candida dubliniensis, Cryptococcus neoformans, Histoplama capsulatum, Aspergillus fumigatus, Coccidiodes immitis, Paracoccidiodes brasiliensis, Blastomyces dermitidis, Pneomocystis camii, Penicillium marneffi, Alternaria alternate.

The microorganism may also be an, or from an, alga, including for example algae that may be, or may have been, classified as protista. Representative algal species include Chaetophora, Chlorella protothecoides, Coleochaete scutata, Coleochaete soluta, Cyanidioschyzon merolae Aphanochaete, Gloeotaenium, Oedogonium, Oocystis, Oscillatoria, Paradoxia multisitia, Phormidium, Chroococcus, Aphanothece, Fragillaria, Cocconis, Navicula, Cymbella, Phaeodactylum as well as cyanobacteria (blue-green algae) and diatoms such as Nitzschia palea.

In one embodiment of this aspect the microorganism is in a biofilm.

To the extent constrained by the above recited range endpoints, in certain embodiments the antimicrobial composition of use in this aspect of the invention will comprise the decylglucoside at a concentration of about 2 mM or greater, e.g. about 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.5 mM, 5 mM, 6 mM, 7 mM, 8 mM, 9 mM, 10 mM, 12 mM, 14 mM, 16 mM, 18 mM, 20 mM, or 30 mM or greater.

In other embodiments, to the extent constrained by the above recited range endpoints, the composition of use in this aspect of the invention will comprise the decylglucoside at a concentration of equal to or less than about 40 mM, e.g. equal to or less than about 30 mM, 20 mM, 18 mM, 16 mM, 14 mM, 12 mM, 10 mM, 9 mM, 8 mM, 7 mM, 6 mM, 5 mM, 4.5 mM, 4 mM, 3.9 mM, 3.8 mM, 3.7 mM, 3.6 mM, 3.5 mM, 3.4 mM, 3.3 mM, 3.2 mM, 3.1 mM, 3 mM, 2.9 mM, 2.8 mM, 2.7 mM, 2.6 mM, 2.5 mM, 2.4 mM, 2.3 mM, 2.2 mM, or 2.1 mM.

Ranges of concentrations with endpoints formed from the combination of any and all of the concentrations recited above are explicitly contemplated. By way of example the antimicrobial composition may comprise the decylglucoside at a concentration of about 2 mM to about 40 mM, e.g. about 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.5 mM, 5 mM, 6 mM, 7 mM, 8 mM, 9 mM, 10 mM, 12 mM, 14 mM, 16 mM, 18 mM, 20 mM, or 30 mM to about 40 mM. In another example the antimicrobial composition may comprise the decylglucoside at a concentration of about 2 mM to about 40 mM, e.g. about 2 mM to about 30 mM, 20 mM, 18 mM, 16 mM, 14 mM, 12 mM, 10 mM, 9 mM, 8 mM, 7 mM, 6 mM, 5 mM, 4.5 mM, 4 mM, 3.9 mM, 3.8 mM, 3.7 mM, 3.6 mM, 3.5 mM, 3.4 mM, 3.3 mM, 3.2 mM, 3.1 mM, 3 mM, 2.9 mM, 2.8 mM, 2.7 mM, 2.6 mM, 2.5 mM, 2.4 mM, 2.3 mM, 2.2 mM, or about 2.1 mM.

To the extent constrained by the above recited range endpoints, in certain embodiments the antimicrobial composition of this aspect of the invention will comprise the at least one C₁ to C₈ carboxylic acid, or a water-soluble salt thereof, at a concentration of about 1 μM or greater, e.g. about 5 μM, 10 μM, 25 μM, 50 μM, 100 μM, 200 μM, 250 μM, 500 μM, 800 μM, 1 mM, 1.6 mM, 2 mM, 2.4 mM, 2.5 mM, 3 mM, 4 mM, 5 mM, 5.6 mM, 6 mM, 7 mM, 8 mM, 9 mM, 10 mM, 20 mM, 30 mM, 40 mM, 50 mM, 60 mM, 70 mM, 80 mM, or 90 mM or greater.

In other embodiments, to the extent constrained by the above recited range endpoints, the antimicrobial composition may comprise the at least one C₁ to C₈ carboxylic acid, or a water-soluble salt thereof, at a concentration of equal to or less than about 100 mM, e.g. equal to or less than about 90 mM, 80 mM, 70 mM, 60 mM, 50 mM, 40 mM, 30 mM, 20 mM, 10 mM, 9 mM, 8 mM, 7 mM, 6 mM, 5.6 mM, 5 mM, 4 mM, 3 mM, 2.5 mM, 2.4 mM, 2 mM, 1.6 mM, 1 mM, 800 μM, 500 μM, 250 μM, 200 μM, 100 μM, 50 μM, 25 μM, 10 μM, or 5 μM, preferably equal to or less than about 10 mM, 9 mM, 8 mM, 7 mM, 6 mM, 5.6 mM, 5 mM, 4 mM, 3 mM, 2.5 mM, 2.4 mM, 2 mM, 1.6 mM, 1 mM, 800 μM, 500 μM, 250 μM, 200 μM, 100 μM, 50 μM, 25 μM, 10 μM, or 5 μM, more preferably equal to or less than about 2.5 mM, 2.4 mM, 2 mM, 1.6 mM, 1 mM, 800 μM, 500 μM, 250 μM, 200 μM, 100 μM, 50 μM, 25 μM, 10 μM, or 5 μM.

Ranges of concentrations with endpoints formed from the combination of any and all of the concentrations recited above are explicitly contemplated. By way of example the antimicrobial composition may comprise the C₁ to C₈ carboxylic acid, or a water-soluble salt thereof, at a concentration of about 1 μM to about 100 mM, e.g. about 5 μM, 10 μM, 25 μM, 50 μM, 100 μM, 200 μM, 250 μM, 500 μM, 800 μM, 1 mM, 1.6 mM, 2 mM, 2.4 mM, 2.5 mM, 3 mM, 4 mM, 5 mM, 5.6 mM, 6 mM, 7 mM, 8 mM, 9 mM, 10 mM, 20 mM, 30 mM, 40 mM, 50 mM, 60 mM, 70 mM, 80 mM, or 90 mM to about 100 mM, preferably 1 μM to about 10 mM, e.g. about 5 μM, 10 μM, 25 μM, 50 μM, 100 μM, 200 μM, 250 μM, 500 μM, 800 μM, 1 mM, 1.6 mM, 2 mM, 2.4 mM, 2.5 mM, 3 mM, 4 mM, 5 mM, 5.6 mM, 6 mM, 7 mM, 8 mM or 9 mM, to about 10 mM, more preferably 1 μM to about 2.5 mM, e.g. about 5 μM, 10 μM, 25 μM, 50 μM, 100 μM, 200 μM, 250 μM, 500 μM, 800 μM, 1 mM, 1.6 mM, 2 mM, or 2.4 mM, to about 2.5 mM, more preferably 1 μM to about 1 mM, e.g. about 5 μM, 10 μM, 25 μM, 50 μM, 100 μM, 200 μM, 250 μM, or 500 μM to about 800 μM. In another example the antimicrobial composition may comprise the at least one C₁ to C₈ carboxylic acid, or a water-soluble salt thereof, at a concentration of about 1 μM to about 90 mM, e.g. about 1 μM to about 80 mM, 70 mM, 60 mM, 50 mM, 40 mM, 30 mM, 20 mM, 10 mM, 9 mM, 8 mM, 7 mM, 6 mM, 5.6 mM, 5 mM, 4 mM, 3 mM, 2.4 mM, 2 mM, 1.6 mM, 1 mM, 800 μM, 500 μM, 250 μM, 200 μM, 100 μM, 50 μM, 25 μM, 10 μM, or about 5 μM, preferably about 1 μM to about 10 mM, 9 mM, 8 mM, 7 mM, 6 mM, 5.6 mM, 5 mM, 4 mM, 3 mM, 2.4 mM, 2 mM, 1.6 mM, 1 mM, 800 μM, 500 μM, 250 μM, 200 μM, 100 μM, 50 μM, 25 μM, 10 μM, or about 5 μM, more preferably about 1 μM to about 2.5 mM, 2.4 mM, 2 mM, 1.6 mM, 1 mM, 800 μM, 500 μM, 250 μM, 200 μM, 100 μM, 50 μM, 25 μM, 10 μM, or about 5 μM, more preferably about 1 μM to about 1 mM, 800 μM, 500 μM, 250 μM, 200 μM, 100 μM, 50 μM, 25 μM, 10 μM, or about 5 μM.

The concentrations given for each component are references to the amounts of said components dissolved in the aqueous portion of the composition. Such compositions may be considered “ready to use” compositions.

The antimicrobial composition is aqueous insofar as it contains water in sufficient quantity to dissolve the decylglucoside and the at least one C₁ to C₈ carboxylic acid, or a water-soluble salt thereof, to the required concentrations. In certain embodiments of the antimicrobial compositions of the invention at least 10% w/w of the composition is water, e.g. at least 15%, 20%, 25%, 35%, 40%, 45%, 50%, 55%, 60%, 65%, 70%, 75%, 80%, 85%, 90%, 95% or 99% of the antimicrobial composition is water, allowance being made for the decylglucoside and the at least one C₁ to C₈ carboxylic acid, or a water-soluble salt thereof, and any other excipients, carriers or other active agents which may be present. Preferably the aqueous composition does not contain a non-polar, e.g. organic, solvent, or if it does, the amount of non-polar solvent is less, e.g. significantly less, than the amount of water in the composition. Expressed numerically the amount of non-polar solvent in the composition is preferably less than 50%, 30%, 20%, 10%, 5%, or 1% of the amount of water solvent in the composition. Solvents with a dielectric constant of less than 15, e.g. less than 12, 10, 8 or 5, are generally considered to be non-polar.

The aqueous antimicrobial compositions may comprise further components, e.g. excipients, carriers or other active agents (in particular other antimicrobial agents or agents which enhance surface adherence of the components of the composition). For completeness it should be made clear that these excipients, carriers or other active agents are not a decylglucoside or a C₁ to C₈ carboxylic acid, or water-soluble salt thereof, or water. As the above mentioned components are biodegradable and non-hazardous to humans and other higher organisms (mammals, birds, fish, reptiles, insects and plants) any further components are preferably also biodegradable and non-hazardous to humans and other higher organisms.

In the context of the present invention “biodegradable” means that the components of the compositions of the invention and thus the compositions themselves degrade in the environment given sufficient time, e.g. a month, 6 months, a year, 2 years or 5 years.

In the context of the invention “non-hazardous to humans and other higher organisms” means that a substance, in the amount at which it is intended for use or in amounts that may typically accumulate following such use, is not harmful to the health or well-being of humans and other higher organisms. Such substances may be selected from those substances Generally Regarded as Safe under sections 201(s) and 409 of the Federal Food, Drug, and Cosmetic Act and/or those listed as safe under relevant local biocide ordinance (e.g. the EU Biocide Regulation No 528/2012).

However, in further preferred embodiments the antimicrobial aqueous composition consists substantially, e.g. essentially, of a decylglucoside at a concentration of about 2 mM to about 40 mM, at least one C₁ to C₈ carboxylic acid, or a water-soluble salt thereof, at a concentration of about 1 μM to about 100 mM, and water, i.e. any further excipients, carriers or other active agents are present in negligible amounts. This may be expressed as an antimicrobial aqueous composition consisting of the following:
(i) a decylglucoside at a concentration of about 2 mM to about 40 mM,
(ii) at least one C₁ to C₈ carboxylic acid, or a water-soluble salt thereof, at a concentration of about 1 μM to about 100 mM,
(iii) equal or less than about 10% w/w (e.g. equal or less than about 9%, 8%, 7%, 6%, 5%, 4%, 3%, 2%, 1%, 0.5%, 0.1%, or 0.01% w/w) in total of excipients, carriers or active agents other than water, and
(iv) an amount of water sufficient to provide a total percentage w/w of 100%.

In other embodiments the antimicrobial aqueous composition consists of water and the following dissolved therein:
(i) a decylglucoside at a concentration of about 2 mM to about 40 mM,
(ii) at least one C₁ to C₈ carboxylic acid, or a water-soluble salt thereof, at a concentration of about 1 μM to about 100 mM.

In these embodiments it is preferred that the at least one C₁ to C₈ carboxylic acid, or water soluble salt thereof, is lactic acid, or water soluble salt thereof, and/or citric acid, or water soluble salt thereof.

In other embodiments the antimicrobial aqueous composition consists of a decylglucoside, lactic acid, or water soluble salt thereof, and water, wherein:
(i) the decylglucoside is present at a concentration of about 300 ppm to about 3000 ppm,
(ii) the lactic acid, or water soluble salt thereof, is present at a concentration of about 0.1 ppm to about 10000 ppm.

These compositions in particular may be considered ready to use compositions.

To the extent constrained by the above recited range endpoints, the decylglucoside may be present at a concentration of about 300 ppm or greater, e.g. about 310 ppm, 320 ppm, 330 ppm, 340 ppm, 350 ppm, 360 ppm, 370 ppm, 380 ppm, 390 ppm, 400 ppm, 410 ppm, 420 ppm, 430 ppm, 440 ppm, 450 ppm, 460 ppm, 470 ppm, 480 ppm, 490 ppm, 500 ppm, 550 ppm, 600 ppm, 700 ppm, 800 ppm, 900 ppm, 1000 ppm, 1100 ppm, 1200 ppm, 1400 ppm, 1600 ppm, 1800 ppm, 2000 ppm, or 2500 ppm or greater.

To the extent constrained by the above recited range endpoints, the decylglucoside may be present at a concentration of equal to or less than about 3000 ppm, e.g. equal to or less than about 2500 ppm, 2000 ppm, 1800 ppm, 1600 ppm, 1400 ppm, 1200 ppm, 1100 ppm, 1000 ppm, 900 ppm, 800 ppm, 700 ppm, 600 ppm, 550 ppm, 500 ppm, 490 ppm, 480 ppm, 470 ppm, 460 ppm, 450 ppm, 440 ppm, 430 ppm, 420 ppm, 410 ppm, 400 ppm, 390 ppm, 380 ppm, 370 ppm, 360 ppm, 350 ppm, 340 ppm, 330 ppm, 320 ppm, or 310 ppm.

Ranges of concentrations with endpoints formed from the combination of any and all of the concentrations recited above are explicitly contemplated. By way of example the decylglucoside may be present at a concentration of about 300 ppm to about 3000 ppm, e.g. about 310 ppm, 320 ppm, 330 ppm, 340 ppm, 350 ppm, 360 ppm, 370 ppm, 380 ppm, 390 ppm, 400 ppm, 410 ppm, 420 ppm, 430 ppm, 440 ppm, 450 ppm, 460 ppm, 470 ppm, 480 ppm, 490 ppm, 500 ppm, 550 ppm, 600 ppm, 700 ppm, 800 ppm, 900 ppm, 1000 ppm, 1100 ppm, 1200 ppm, 1400 ppm, 1600 ppm, 1800 ppm, 2000 ppm or 2500 ppm to about 3000 ppm.

In another example the decylglucoside may be present at a concentration of about 300 ppm to about 2500 ppm, e.g. about 300 ppm to about 2000 ppm, 1800 ppm, 1600 ppm, 1400 ppm, 1200 ppm, 1100 ppm, 1000 ppm, 900 ppm, 800 ppm, 700 ppm, 600 ppm, 550 ppm, 500 ppm, 490 ppm, 480 ppm, 470 ppm, 460 ppm, 450 ppm, 440 ppm, 430 ppm, 420 ppm, 410 ppm, 400 ppm, 390 ppm, 380 ppm, 370 ppm, 360 ppm, 350 ppm, 340 ppm, 330 ppm, 320 ppm, or 310 ppm.

To the extent constrained by the above recited range endpoints, the lactic acid, or water soluble salt thereof, may be present at a concentration of about at a concentration of about 0.1 ppm or greater, e.g. about 0.05 ppm, 1 ppm, 2.5 ppm, 5 ppm, 10 ppm, 20 ppm, 25 ppm, 50 ppm, 80 ppm, 100 ppm, 160 ppm, 200 ppm, 240 ppm, 250 ppm, 300 ppm, 400 ppm, 500 ppm, 560 ppm, 600 ppm, 700 ppm, 800 ppm, 900 ppm, 1000 ppm, 2000 ppm, 3000 ppm, 4000 ppm, 5000 ppm, 6000 ppm, 7000 ppm, 8000 ppm, or 9000 ppm or greater.

To the extent constrained by the above recited range endpoints, the lactic acid, or water soluble salt thereof, may be present at a concentration of equal to or less than about 10000 ppm, e.g. equal to or less than about 9000 ppm, 8000 ppm, 7000 ppm, 6000 ppm, 5000 ppm, 4000 ppm, 3000 ppm, 2000 ppm, 1000 ppm, 900 ppm, 800 ppm, 700 ppm, 600 ppm, 560 ppm, 500 ppm, 400 ppm, 300 ppm, 250 ppm, 240 ppm, 200 ppm, 160 ppm, 100 ppm, 80 ppm, 50 ppm, 25 ppm, 20 ppm, 10 ppm, 5 ppm, 2.5 ppm, 1 ppm, or 0.5 ppm, preferably equal to or less than about 1000 ppm, 900 ppm, 800 ppm, 700 ppm, 600 ppm, 560 ppm, 500 ppm, 400 ppm, 300 ppm, 250 ppm, 240 ppm, 200 ppm, 160 ppm, 100 ppm, 80 ppm, 50 ppm, 25 ppm, 20 ppm, 10 ppm, 5 ppm, 2.5 ppm, 1 ppm, or 0.5 ppm, more preferably equal to or less than about 250 ppm, 240 ppm, 200 ppm, 160 ppm, 100 ppm, 80 ppm, 50 ppm, 25 ppm, 20 ppm, 10 ppm, 5 ppm, 2.5 ppm, 1 ppm, or 0.5 ppm, more preferably equal to or less than about 100 ppm, 80 ppm, 50 ppm, 25 ppm, 20 ppm, 10 ppm, 5 ppm, 2.5 ppm, 1 ppm, or 0.5 ppm.

Ranges of concentrations with endpoints formed from the combination of any and all of the concentrations recited above are explicitly contemplated. By way of example, in these embodiments the lactic acid, or water soluble salt thereof, may be present at a concentration of about 0.1 ppm to about 10000 ppm, e.g. about 0.5 ppm, 1 ppm, 2.5 ppm, 5 ppm, 10 ppm, 20 ppm, 25 ppm, 50 ppm, 80 ppm, 100 ppm, 160 ppm, 200 ppm, 240 ppm, 250 ppm, 300 ppm, 400 ppm, 500 ppm, 560 ppm, 600 ppm, 700 ppm, 800 ppm, 900 ppm, 1000 ppm, 2000 ppm, 3000 ppm, 4000 ppm, 5000 ppm, 6000 ppm, 7000 ppm, 8000 ppm or 9000 ppm to about 10000 ppm, preferably about 0.1 ppm to about 1000 ppm, e.g. about 0.5 ppm, 1 ppm, 2.5 ppm, 5 ppm, 10 ppm, 20 ppm, 25 ppm, 50 ppm, 80 ppm, 100 ppm, 160 ppm, 200 ppm, 240 ppm, 250 ppm, 300 ppm, 400 ppm, 500 ppm, 560 ppm, 600 ppm, 700 ppm, 800 ppm or 900 ppm to about 1000 ppm; more preferably 0.1 ppm to about 250 ppm, e.g. about 0.5 ppm, 1 ppm, 2.5 ppm, 5 ppm, 10 ppm, 20 ppm, 25 ppm, 50 ppm, 80 ppm, 100 ppm, 160 ppm, 200 ppm or 240 ppm to about 250 ppm; more preferably about 0.1 ppm to about 100 ppm, e.g. about 0.5 ppm, 1 ppm, 2.5 ppm, 5 ppm, 10 ppm, 20 ppm, 25 ppm, 50 ppm or 80 ppm to about 100 ppm.

In another example, in these embodiments the lactic acid, or water soluble salt thereof, may be present at a concentration of about 0.1 ppm to about 9000 ppm, e.g. about 0.1 ppm to about 8000 ppm, 7000 ppm, 6000 ppm, 5000 ppm, 4000 ppm, 3000 ppm, 2000 ppm, 1000 ppm, 900 ppm, 800 ppm, 700 ppm, 600 ppm, 560 ppm, 500 ppm, 400 ppm, 300 ppm, 250 ppm, 240 ppm, 200 ppm, 160 ppm, 100 ppm, 80 ppm, 50 ppm, 25 ppm, 20 ppm, 10 ppm, 5 ppm, 2.5 ppm, 1 ppm, or 0.5 ppm, more preferably about 0.1 ppm to about 1000 ppm, 900 ppm, 800 ppm, 700 ppm, 600 ppm, 560 ppm, 500 ppm, 400 ppm, 300 ppm, 250 ppm, 240 ppm, 200 ppm, 160 ppm, 100 ppm, 80 ppm, 50 ppm, 25 ppm, 20 ppm, 10 ppm, 5 ppm, 2.5 ppm, 1 ppm, or 0.5 ppm, more preferably about 0.1 ppm to about 250 ppm, 240 ppm, 200 ppm, 160 ppm, 100 ppm, 80 ppm, 50 ppm, 25 ppm, 20 ppm, 10 ppm, 5 ppm, 2.5 ppm, 1 ppm, or 0.5 ppm, more preferably 0.1 ppm to about 100 ppm, 80 ppm, 50 ppm, 25 ppm, 20 ppm, 10 ppm, 5 ppm, 2.5 ppm, 1 ppm, or 0.5 ppm.

These compositions in particular may be considered ready to use compositions.

In other embodiments the antimicrobial aqueous composition consists of a decylglucoside, citric acid, or water soluble salt thereof, and water, wherein:
(i) the decylglucoside is present at a concentration of about 300 ppm to about 3000 ppm,
(ii) the citric acid, or water soluble salt thereof, is present at a concentration of about 0.2 ppm to about 20000 ppm.

The above recited ppm values for the concentration of decylglucoside apply mutatis mutandis.

To the extent constrained by the above recited range endpoints, in these embodiments the citric acid, or water soluble salt thereof, may be present at a concentration of about 0.2 ppm or greater, e.g. 0.5 ppm, 1 ppm, 2.5 ppm, 5 ppm, 10 ppm, 20 ppm, 25 ppm, 50 ppm, 80 ppm, 100 ppm, 160 ppm, 200 ppm, 240 ppm, 250 ppm, 300 ppm, 400 ppm, 500 ppm, 560 ppm, 600 ppm, 700 ppm, 800 ppm, 900 ppm, 1000 ppm, 2000 ppm, 3000 ppm, 4000 ppm, 5000 ppm, 6000 ppm, 7000 ppm, 8000 ppm, 9000 ppm, 10000 ppm, or 15000 ppm or greater.

To the extent constrained by the above recited range endpoints, in these embodiments the citric acid, or water soluble salt thereof, may be present at a concentration of equal to or less than about 20000 ppm, e.g. equal to or less than about 15000 ppm, 10000 ppm, 9000 ppm, 8000 ppm, 7000 ppm, 6000 ppm, 5000 ppm, 4000 ppm, 3000 ppm, 2000 ppm, 1000 ppm, 900 ppm, 800 ppm, 700 ppm, 600 ppm, 560 ppm, 500 ppm, 400 ppm, 300 ppm, 250 ppm, 240 ppm, 200 ppm, 160 ppm, 100 ppm, 80 ppm, 50 ppm, 25 ppm, 20 ppm, 10 ppm, 5 ppm, 2.5 ppm, 1 ppm, or 0.5 ppm, preferably equal to or less than about 1000 ppm, 900 ppm, 800 ppm, 700 ppm, 600 ppm, 560 ppm, 500 ppm, 400 ppm, 300 ppm, 250 ppm, 240 ppm, 200 ppm, 160 ppm, 100 ppm, 80 ppm, 50 ppm, 25 ppm, 20 ppm, 10 ppm, 5 ppm, 2.5 ppm, 1 ppm, or 0.5 ppm, more preferably equal to or less than about 250 ppm, 240 ppm, 200 ppm, 160 ppm, 100 ppm, 80 ppm, 50 ppm, 25 ppm, 20 ppm, 10 ppm, 5 ppm, 2.5 ppm, 1 ppm, or 0.5 ppm, more preferably equal to or less than about 100 ppm, 80 ppm, 50 ppm, 25 ppm, 20 ppm, 10 ppm, 5 ppm, 2.5 ppm, 1 ppm, or 0.5 ppm.

Ranges of concentrations with endpoints formed from the combination of any and all of the concentrations recited above are explicitly contemplated. By way of example, in these embodiments the citric acid, or water soluble salt thereof, may be present at a concentration of about 0.2 ppm to about 20000 ppm, e.g. about 0.5 ppm, 1 ppm, 2.5 ppm, 5 ppm, 10 ppm, 20 ppm, 25 ppm, 50 ppm, 80 ppm, 100 ppm, 160 ppm, 200 ppm, 240 ppm, 250 ppm, 300 ppm, 400 ppm, 500 ppm, 560 ppm, 600 ppm, 700 ppm, 800 ppm, 900 ppm, 1000 ppm, 2000 ppm, 3000 ppm, 4000 ppm, 5000 ppm, 6000 ppm, 7000 ppm, 8000 ppm, 9000 ppm, 10000 ppm or 15000 ppm to about 20000 ppm, preferably about 0.2 ppm to about 1000 ppm, e.g. about 0.5 ppm, 1 ppm, 2.5 ppm, 5 ppm, 10 ppm, 20 ppm, 25 ppm, 50 ppm, 80 ppm, 100 ppm, 160 ppm, 200 ppm, 240 ppm, 250 ppm, 300 ppm, 400 ppm, 500 ppm, 560 ppm, 600 ppm, 700 ppm, 800 ppm, 900 ppm to about 1000 ppm, more preferably about 0.2 ppm to about 250 ppm, e.g. about 0.5 ppm, 1 ppm, 2.5 ppm, 5 ppm, 10 ppm, 20 ppm, 25 ppm, 50 ppm, 80 ppm, 100 ppm, 160 ppm, 200 ppm or 240 ppm to about 250 ppm, more preferably about 0.2 ppm to about 100 ppm, e.g. about 0.5 ppm, 1 ppm, 2.5 ppm, 5 ppm, 10 ppm, 20 ppm, 25 ppm, 50 ppm, or 80 ppm to about 100 ppm.

In another example, in these embodiments the citric acid, or water soluble salt thereof, may be present at a concentration of about 0.2 ppm to about 15000 ppm, e.g. about 0.2 ppm to about 10000 ppm, 9000 ppm, 8000 ppm, 7000 ppm, 6000 ppm, 5000 ppm, 4000 ppm, 3000 ppm, 2000 ppm, 1000 ppm, 900 ppm, 800 ppm, 700 ppm, 600 ppm, 560 ppm, 500 ppm, 400 ppm, 300 ppm, 250 ppm, 240 ppm, 200 ppm, 160 ppm, 100 ppm, 80 ppm, 50 ppm, 25 ppm, 20 ppm, 10 ppm, 5 ppm, 2.5 ppm, 1 ppm, or 0.5 ppm, more preferably about 0.2 ppm to about 1000 ppm, 900 ppm, 800 ppm, 700 ppm, 600 ppm, 560 ppm, 500 ppm, 400 ppm, 300 ppm, 250 ppm, 240 ppm, 200 ppm, 160 ppm, 100 ppm, 80 ppm, 50 ppm, 25 ppm, 20 ppm, 10 ppm, 5 ppm, 2.5 ppm, 1 ppm, or 0.5 ppm, more preferably about 0.2 ppm to about 250 ppm, 240 ppm, 200 ppm, 160 ppm, 100 ppm, 80 ppm, 50 ppm, 25 ppm, 20 ppm, 10 ppm, 5 ppm, 2.5 ppm, 1 ppm, or 0.5 ppm, more preferably about 0.2 ppm to about 100 ppm, 80 ppm, 50 ppm, 25 ppm, 20 ppm, 10 ppm, 5 ppm, 2.5 ppm, 1 ppm, or 0.5 ppm.

These compositions in particular may be considered ready to use compositions.

In other embodiments the antimicrobial aqueous composition consists of a decylglucoside, citric acid, or water soluble salt thereof, lactic acid, or water soluble salt thereof, and water wherein:
(i) the decylglucoside is present at a concentration of about 300 ppm to about 3000 ppm,
(ii) the lactic acid, or water soluble salt thereof, is present at a concentration of about 0.05 ppm to about 5000 ppm.
(iii) the citric acid, or water-soluble salt thereof, is present at a concentration of about 0.1 ppm to about 10000 ppm.

The above recited ppm values for the concentration of decylglucoside apply mutatis mutandis.

The ppm values for the concentration of lactic acid, or water soluble salt thereof, and citric acid, or water soluble salt thereof may be derived from those recited above by halving said respective values.

These compositions in particular may be considered ready to use compositions.

Of particular note as further components of the compositions of use in accordance with this aspect of the invention are the octylglucosides. The octylglucosides are a class of alkyl glycoside containing monomeric glucose as the glycone group and an 8 carbon saturated alkyl chain linked to the glucose residue via a glycosidic bond, e.g. an S-, N-, C-, or O-glycosidic bond. Preferably the octylcoside is octyl β-D-glucopyranoside. For completeness it should be made clear that in these embodiments “excipients, carriers or other active agents” are not a decylglucoside, an octylglucoside or a C₁ to C₈ carboxylic acid, or water-soluble salt thereof, or water.

The octylglucoside, e.g. octyl β-D-glucopyranoside may be present in the aqueous composition of the invention in a ratio (which may be calculated on volume by volume, or preferably a weight by weight, basis) of octylglucoside to decylglucoside of at least about 10:100, 10:90, 15:85, 20:80, 25:75, 30:70, 35:65, 40:60, 45:55, 50:50, 55:45, 60:40, 65:35, 70:30, 75:25, 80:20, 85:15, 90:10 or 100:10. Typically, the octylglucoside to decylglucoside ratio will be no more than about, e.g. about 100:10, 90:10, 85:15, 80:20, 75:25, 70:30, 65:35, 60:40, 55:45, 50:50, 45:55, or 40:60. A ratio of 35-45 octyl to 65-55 decyl, e.g. 37-43 octyl to 63-57 decyl, 38-42 octyl to 62-58 decyl, 39-41 octyl to 61-59 decyl or about 40 octyl to about 60 decyl are advantageous in accordance with the invention. A mixture of these compounds in said proportions is available from commercial suppliers.

Thus in further embodiments the antimicrobial aqueous composition may comprise:
(i) a decylglucoside at a concentration of about 2 mM to about 40 mM,
(ii) an octylglucoside at a concentration of about 0.2 mM (about 30 ppm) to about 200 mM, preferably wherein the ratio of said octylglucoside to said decylglucoside is at least about 10:100, and
(iii) at least one C₁ to C₈ carboxylic acid, or a water-soluble salt thereof, at a concentration of about 1 μM to about 100 mM.

To the extent constrained by the above recited range endpoints, in certain embodiments the composition of the invention may comprise the octylglucoside at a concentration of about 0.2 mM or greater, e.g. about 0.21 mM, 0.22 mM, 0.23 mM, 0.24 mM, 0.25 mM, 0.26 mM, 0.27 mM, 0.28 mM, 0.29 mM, 0.3 mM, 0.31 mM, 0.32 mM, 0.33 mM, 0.34 mM, 0.35 mM, 0.36 mM, 0.37 mM, 0.38 mM, 0.39 mM, 0.4 mM, 0.45 mM, 0.5 mM, 0.6 mM, 0.7 mM, 0.8 mM, 0.9 mM, 1 mM, 1.2 mM, 1.4 mM, 1.6 mM, 1.8 mM, 2 mM, 3 mM, 4 mM, 4.5 mM, 5 mM, 6 mM, 7 mM, 8 mM, 9 mM, 10 mM, 12 mM, 14 mM, 16 mM, 18 mM, 20 mM, 30 mM, 40 mM, 50 mM, 60 mM, 80 mM, 100 mM, 150 mM, 200 mM, or greater.

To the extent constrained by the above recited range endpoints, in other embodiments the composition of the invention as used to combat microorganisms (which may be considered a “ready to use” composition) may comprise the octylglucoside at a concentration of equal to or less than about 200 mM, e.g. equal to or less than about 150 mM, 100 mM, 80 mM, 60 mM, 50 mM, 40 mM, 30 mM, 20 mM, 18 mM, 16 mM, 14 mM, 12 mM, 10 mM, 9 mM, 8 mM, 7 mM, 6 mM, 5 mM, 4 mM, 3 mM, 2 mM, 1.8 mM, 1.6 mM, 1.4 mM, 1.2 mM, 1 mM, 0.9 mM, 0.8 mM, 0.7 mM, 0.6 mM, 0.5 mM, 0.45 mM, 0.4 mM, 0.39 mM, 0.38 mM, 0.37 mM, 0.36 mM, 0.35 mM, 0.34 mM, 0.33 mM, 0.32 mM, 0.3 mM, 0.29 mM, 0.28 mM, 0.27 mM, 0.26 mM, 0.25 mM, 0.24 mM, 0.23 mM, 0.22 mM, or about 0.21 mM.

Ranges of concentrations with endpoints formed from the combination of any and all of the concentrations recited above are explicitly contemplated. By way of example the composition of the invention as used to combat microorganisms may comprise the octylglucoside at a concentration of about 0.2 mM to about 200 mM, e.g. about 0.21 mM 0.22 mM, 0.23 mM, 0.24 mM, 0.25 mM, 0.26 mM, 0.27 mM, 0.28 mM, 0.29 mM, 0.3 mM, 0.31 mM, 0.32 mM, 0.33 mM, 0.34 mM, 0.35 mM, 0.36 mM, 0.37 mM, 0.38 mM, 0.39 mM, 0.4 mM, 0.45 mM, 0.5 mM, 0.6 mM, 0.7 mM, 0.8 mM, 0.9 mM, 1 mM, 1.2 mM, 1.4 mM, 1.6 mM, 1.8 mM, 2 mM, 3 mM, 4 mM, 4.5 mM, 5 mM, 6 mM, 7 mM, 8 mM, 9 mM, 10 mM, 12 mM, 14 mM, 16 mM, 18 mM, 20 mM, 30 mM, 40 mM, 50 mM, 60 mM, 80 mM, 100 mM, 150 mM to about 200 mM. In another example the composition of the invention as used to combat microorganisms may comprise the octylglucoside at a concentration of about 0.2 mM to about 150 mM, e.g. about 0.2 mM to about 100 mM, 80 mM, 60 mM, 50 mM, 40 mM, 30 mM, 20 mM, 18 mM, 16 mM, 14 mM, 12 mM, 10 mM, 9 mM, 8 mM, 7 mM, 6 mM, 5 mM, 4 mM, 3 mM, 2 mM, 1.8 mM, 1.6 mM, 1.4 mM, 1.2 mM, 1 mM, 0.9 mM, 0.8 mM, 0.7 mM, 0.6 mM, 0.5 mM, 0.45 mM, 0.4 mM, 0.39 mM, 0.38 mM, 0.37 mM, 0.36 mM, 0.35 mM, 0.34 mM, 0.33 mM, 0.32 mM, 0.3 mM, 0.29 mM, 0.28 mM, 0.27 mM, 0.26 mM, 0.25 mM, 0.24 mM, 0.23 mM, 0.22 mM or about 0.21 mM.

These compositions in particular may be considered ready to use compositions.

In further preferred embodiments the antimicrobial aqueous composition consists substantially, e.g. essentially, of a decylglucoside at a concentration of about 2 mM to about 40 mM, an octylglucoside at a concentration of about 0.2 mM to about 200 mM, wherein the ratio of said octylglucoside to said decylglucoside is at least about 10:100, at least one C₁ to C₈ carboxylic acid, or a water-soluble salt thereof, at a concentration of about 1 μM to about 100 mM, and water, i.e. any excipients, carriers or other active agents are present in negligible amounts.

This may be expressed as an antimicrobial aqueous composition consisting of the following:
(i) a decylglucoside at a concentration of about 2 mM to about 40 mM,
(ii) an octylglucoside at a concentration of about 0.2 mM to about 200 mM, preferably wherein the ratio of said octylglucoside to said decylglucoside is at least 10:100,
(iii) at least one C₁ to C₈ carboxylic acid, or a water-soluble salt thereof, at a concentration of about 1 μM to about 100 mM,
(iv) equal or less than about 10% w/w (e.g. equal or less than about 9%, 8%, 7%, 6%, 5%, 4%, 3%, 2%, 1%, 0.5%, 0.1%, or 0.01% w/w) in total of excipients, carriers or active agents other than water, and
(v) an amount of water sufficient to provide a total percentage w/w of 100%.

In other embodiments the antimicrobial aqueous composition consists of water and the following dissolved therein:
(i) a decylglucoside at a concentration of about 2 mM to about 40 mM,
(ii) an octylglucoside at a concentration of about 0.2 mM to about 200 mM, preferably wherein the ratio of said octylglucoside to said decylglucoside is at least 10:100, and
(iii) at least one C₁ to C₈ carboxylic acid, or a water-soluble salt thereof, at a concentration of about 1 μM to about 100 mM.

These compositions in particular may be considered ready to use compositions.

In these embodiments it is preferred that the at least one C₁ to C₈ carboxylic acid, or water soluble salt thereof, is lactic acid, or water soluble salt thereof, and/or citric acid, or water soluble salt thereof.

In other embodiments the antimicrobial aqueous composition consists of a decylglucoside, an octylglucoside, lactic acid, or water soluble salt thereof, and water, wherein:
(i) the decylglucoside is present at a concentration of about 300 ppm to about 3000 ppm,
(ii) the octylglucoside is present at a concentration of about 30 ppm to 3000 ppm, preferably wherein the ratio of said octylglucoside to said decylglucoside is at least 10:100, and
(iii) the lactic acid, or water soluble salt thereof, is present at a concentration of about 0.1 ppm to about 10000 ppm.

To the extent constrained by the above recited range endpoints, in these embodiments the octylglucoside may be present at a concentration of about 31 ppm or greater, e.g. about 32 ppm, 33 ppm, 34 ppm, 35 ppm, 36 ppm, 37 ppm, 38 ppm, 39 ppm, 40 ppm, 41 ppm, 42 ppm, 43 ppm, 44 ppm, 45 ppm, 46 ppm, 47 ppm, 48 ppm, 49 ppm, 50 ppm, 55 ppm, 60 ppm, 70 ppm, 80 ppm, 90 ppm, 100 ppm, 110 ppm, 120 ppm, 140 ppm, 160 ppm, 180 ppm, 200 ppm, 250 ppm, 300 ppm, 400ppm, 450 ppm, 600 ppm, 750 ppm, 900 ppm, 1050 ppm, 1200 ppm, 1350 ppm, 1500 ppm, 1800 ppm, 2100 ppm, 2400 ppm or 2700 ppm or greater.

To the extent constrained by the above recited range endpoints, in other embodiments the octylglucoside may be present at a concentration of equal to or less than about 3000 ppm, e.g. equal to or less than about 2700 ppm, 2400 ppm, 2100 ppm, 1800 ppm, 1500 ppm, 1350 ppm, 1200 ppm, 1050 ppm, 900 ppm, 750 ppm, 600 ppm, 450 ppm, 400ppm, 300 ppm, 250 ppm, 200 ppm, 180 ppm, 160 ppm, 140 ppm, 120 ppm, 110 ppm, 100 ppm, 90 ppm, 80 ppm, 70 ppm, 60 ppm, 55 ppm, 50 ppm, 49 ppm, 48 ppm, 47 ppm, 46 ppm, 45 ppm, 44 ppm, 43 ppm, 42 ppm, 41 ppm, 40 ppm, 39 ppm, 38 ppm, 37 ppm, 36 ppm, 35 ppm, 34 ppm, 33 ppm, 32 ppm, or about 31 ppm.

Ranges of concentrations with endpoints formed from the combination of any and all of the concentrations recited above are explicitly contemplated. By way of example the octylglucoside may be present at a concentration of about 30 ppm to about 3000 ppm, e.g. about 31 ppm, 32 ppm, 33 ppm, 34 ppm, 35 ppm, 36 ppm, 37 ppm, 38 ppm, 39 ppm, 40 ppm, 41 ppm, 42 ppm, 43 ppm, 44 ppm, 45 ppm, 46 ppm, 47 ppm, 48 ppm, 49 ppm, 50 ppm, 55 ppm, 60 ppm, 70 ppm, 80 ppm, 90 ppm, 100 ppm, 110 ppm, 120 ppm, 140 ppm, 160 ppm, 180 ppm, 200 ppm, 250 ppm, 300 ppm, 400ppm, 450 ppm, 600 ppm, 750 ppm, 900 ppm, 1050 ppm, 1200 ppm, 1350 ppm, 1500 ppm, 1800 ppm, 2100 ppm, 2400 ppm or 2700 ppm to about 3000 ppm.

In another example the octylglucoside may be present at a concentration of about 30 ppm to about 2700 ppm, e.g. about 30 ppm to about 2400 ppm, 2100 ppm, 1800 ppm, 1500 ppm, 1350 ppm, 1200 ppm, 1050 ppm, 900 ppm, 750 ppm, 600 ppm, 450 ppm, 400ppm, 300 ppm, 250 ppm, 200 ppm, 180 ppm, 160 ppm, 140 ppm, 120 ppm, 110 ppm, 100 ppm, 90 ppm, 80 ppm, 70 ppm, 60 ppm, 55 ppm, 50 ppm, 49 ppm, 48 ppm, 47 ppm, 46 ppm, 45 ppm, 44 ppm, 43 ppm, 42 ppm, 41 ppm, 40 ppm, 39 ppm, 38 ppm, 37 ppm, 36 ppm, 35 ppm, 34 ppm, 33 ppm, 32 ppm, or about 31 ppm.

These compositions is particular may be considered ready to use compositions.

The above recited ppm values for the concentrations of decylglucoside and lactic acid, or water soluble salt thereof, apply mutatis mutandis.

In other embodiments the invention provides an antimicrobial aqueous composition consisting of a decylglucoside, an octylglucoside, citric acid, or water soluble salt thereof, and water, wherein:
(i) the decylglucoside is present at a concentration of about 300 ppm to about 3000 ppm,
(ii) the octylglucoside is present at a concentration of about 3 ppm to about 3000 ppm, preferably wherein the ratio of said octylglucoside to said decylglucoside is at least about 10:100, and
(iii) the citric acid, or water soluble salt thereof, is present at a concentration of about 0.2 ppm to about 20000 ppm.

The above recited ppm values for the concentrations of decylglucoside, octylglucoside and citric acid, or water soluble salt thereof, apply mutatis mutandis.

In other embodiments the invention provides an antimicrobial aqueous composition consisting of a decylglucoside, an octylglucoside, citric acid, or water soluble salt thereof, lactic acid, or water soluble salt thereof, and water wherein:
(i) the decylglucoside is present at a concentration of about 300 ppm to about 3000 ppm,
(ii) the octylglucoside is present at a concentration of about 30 ppm to about 3000 ppm, preferably wherein the ratio of said octylglucoside to said decylglucoside is at least about 10:100,
(iii) the lactic acid, or water soluble salt thereof, is present at a concentration of about 0.05 ppm to about 5000 ppm,
(iv) the citric acid, or water soluble salt thereof, is present at a concentration of about 0.1 ppm to about 10000 ppm,

The above recited ppm values for the concentrations of decylglucoside, octylglucoside, lactic acid, or water soluble salt thereof, and citric acid, or water soluble salt thereof, apply mutatis mutandis.

In these specific embodiments a ratio of octylglucoside to decylglucoside of about 40:60, e.g. 35-45 octyl to 65-55 decyl, e.g. 37-43 octyl to 63-57 decyl, 38-42 octyl to 62-58 decyl or 39-41 octyl to 61-59 decyl, is preferred.

These compositions in particular may be considered ready to use compositions.

In particular embodiments the (ready to use) antimicrobial aqueous composition consists of a decylglucoside, an octylglucoside, lactic acid, or water soluble salt thereof, and water wherein:
(i) the decylglucoside is present at a concentration of about 600 ppm
(ii) the octylglucoside is present at a concentration of about 400 ppm
(iii) the lactic acid, or water soluble salt thereof, is present at a concentration of about 200 ppm (2 mM).

The pH of the aqueous antimicrobial composition is not limited, although in preferred embodiments the pH is, or is adjusted to be, approximately netural, e.g. a pH of 5 to 9, 6 to 8 or about 7.

The aqueous antimicrobial composition may contain further surfactants (i.e. surfactants other than a decylglucoside and, if present, an octylglucoside, e.g. decyl β-D-glucopyranoside and/or octyl β-D-glucopyranoside) e.g. anionic, cationic or zwitterionic (amphoteric) surfactants or a further non-ionic surfactant.

The further non-ionic surfactant may be selected from polyoxyethylene glycol alkyl ethers (CH₃—(CH₂)₁₀₋₁₆—(O—C₂H₄)₁₋₂₅—OH, e.g. octaethylene glycol monododecyl ether and pentaethylene glycol monododecyl ether), polyoxypropylene glycol alkyl ethers (CH₃—(CH₂)₁₀₋₁₆—(O—C₃H₆)₁₋₂₅—OH), glycoside alkyl ethers (also known as alkyl (poly)glycosides: CH₃—(CH₂)₄₋₂₀-(glycone)₁₋₃-OH, e.g. lauryl glucoside, decyl maltopyranoside, octyl maltopyranoside, octyl thioglucopyranoside, n-heptyl thioglucopyranoside), acyl-N-methyl glucamides (e.g. MEGA 8, 9 (N-nonanoyl-N-methylglucamine) and 10), polyoxyethylene glycol octylphenol ethers (C₈H₁₇—(C₆H₄)—(O—C₂H₄)₁₋₂₅—OH, e.g. Triton X-100), polyoxyethylene glycol alkylphenol ethers (C₉H₁₉—(C₆H₄)—(O—C₂H₄)₁₋₂₅—OH, e.g. Nonoxynol-9), glycerol alkyl esters (e.g. glyceryl laurate), polyoxyethylene glycol sorbitan alkyl esters (e.g. polysorbate), sorbitan alkyl esters (e.g. Spans), cocamide MEA, cocamide DEA, dodecyldimethylamine oxide, block copolymers of polyethylene glycol and polypropylene glycol (e.g. poloxamers), polyethoxylated tallow amine (POEA). In certain embodiments the non-ionic surfactant is not an antimicrobial non-ionic surfactant. In certain embodiments the non-ionic surfactant is not undecenyl monoglucoside or C_{12/14} alkyl oligoglucoside (degree of oligomerization 1.4), of which the alkyl radicals are derived from an n-dodecanol/n-tetradecanol mixture in a ratio by weight of 70:30.

In other embodiments the aqueous compositions of the invention preferably do not comprise alkylglycosides (including alkyl polyglycosides) other than the decylglucoside and, if present, the octylglucoside (e.g. decyl β-D-glucopyranoside and/or octyl β-D-glucopyranoside).

The anionic surfactant may be selected from sulfate, sulfonate, phosphate and carboxylate alkyl esters, preferably alkyl sulfates, alkyl phosphates and alkyl carboxylates. Alkyl sulfates may be exemplified by, but not limited to, ammonium lauryl sulfate, sodium lauryl sulfate (SDS; sodium dodecyl sulfate), sodium laureth sulfate (SLES; sodium lauryl ether sulfate) and sodium myreth sulfate. Alkyl carboxylates may be exemplified by, but not limited to, sodium stearate, sodium lauroyl sarcosinate, perfluorononanoate (PFNA) and perfluorooctanoate (PFOA).

The cationic surfactant may be selected from primary, secondary, or tertiary alkyl amines, e.g. octenidine dihydrochloride, and quaternary ammonium surfactants, which may be exemplified by, but not limited to, alkyltrimethylammonium salts (e.g. cetyl trimethylammonium bromide (CTAB, cetyl trimethylammonium chloride (CTAC)) benzalkonium chloride (BAC; also known as alkyl-dimethylbenzyl ammonium chloride), benzethonium chloride cetalkonium chloride, cetylpyridinium chloride, cetrimonium, didecyldimethylammonium chloride (DDQ), dioctadecyldimethylammonium bromide (DODAB) and domiphen bromide. Preferably the cationic surfactant is a quaternary ammonium surfactant, e.g. benzalkonium chloride (preferably benzyl-C12-16-alkyldimethyl chlorides) and didecyldimethylammonium chloride.

The zwitterionic surfactant may be selected from, for example, the alkyl betaines, the alkylamidopropylbetaines, the alkyl aminopropionates, the alkyliminodipropionates and the alkylimidazolines, e.g. lauryldimethylamine-N-oxide, n-dodecyl-N,N-dimethyl-3-ammonio-1-propanesulfonate, n-decyl-N,N-dimethylamine-N-oxide, n-decyl-N,N-dimethylglycine.

In certain embodiments the aqueous composition of the invention does not contain anionic and/or zwitterionic surfactants. In other embodiments the aqueous composition does not contain cationic surfactants that are not quaternary ammonium surfactants, e.g. benzalkonium chloride and didecyldimethylammonium chloride.

In preferred embodiments the further surfactants will not be antimicrobial.

In other preferred embodiments the only surfactants in detectable amounts in the aqueous antimicrobial composition are the decylglucoside and, if present, the octylglucoside (e.g. decyl β-D-glucopyranoside and/or octyl β-D-glucopyranoside). In other words the antimicrobial composition does not comprise further surfactants (i.e. surfactants other than the decylglucoside and, if present, the octylglucoside).

In other embodiments the aqueous compositions may comprise a further anti-microbial agent, i.e. an antimicrobial agent in addition to the antimicrobial combination of a decylglucoside and, if present, an octylglucoside, together with at least one C₁ to C₈ carboxylic acid, or water-soluble salt thereof. Thus, the further anti-microbial agent is not a decylglucoside, an octylglucoside, or a C₁ to C₈ carboxylic acid, or water-soluble salt thereof. The further antimicrobial agent may be selected from the antibiotics, antiseptics, antimicrobial surfactants, antifungals, antivirals, antimicrobial peptides, antimicrobial polyenes, disinfectants, or cleaning or sterilising agents. In these embodiments the antimicrobial agent may be present in amounts below those required for said agent to exert an antimicrobial effect when used alone.

In other embodiments however the aqueous compositions of the invention do not comprise further antimicrobial agents, or at least an antimicrobial amount of an antimicrobial agent, other than the decylglucoside and, if present, the octylglucoside, and the at least one C₁ to C₈ carboxylic acid.

Representative antibiotics include, but are not limited to the aminoglycosides (e.g. amikacin, gentamicin, kanamycin, neomycin, netilmicin, streptomycin, tobramycin); the carbecephems (e.g. loracarbef); the 1st generation cephalosporins (e.g. cefadroxil, cefazolin, cephalexin); 2nd generation cephalosporins (e.g. cefaclor, cefamandole, cephalexin, cefoxitin, cefprozil, cefuroxime); 3rd generation cephalosporins (e.g. cefixime, cefdinir, cefditoren, cefoperazone, cefotaxime, cefpodoxime, ceftazidime, ceftibuten, ceftizoxime, ceftriaxone); 4th generation cephalosporins (e.g. cefepime); the macrolides (e.g. azithromycin, clarithromycin, dirithromycin, erythromycin, troleandomycin); the monobactams (e.g. aztreonam); the penicillins (e.g. amoxicillin, ampicillin, carbenicillin, cloxacillin, dicloxacillin, nafcillin, oxacillin, penicillin G, penicillin V, piperacillin, ticarcillin); the polypeptide antibiotics (e.g. bacitracin, colistin, polymyxin B); the quinolones (e.g. ciprofloxacin, enoxacin, gatifloxacin, levofloxacin, lomefloxacin, moxifloxacin, norfloxacin, ofloxacin, trovafloxacin); the sulfonamides (e.g. mafenide, sulfacetamide, sulfamethizole, sulfasalazine, sulfisoxazole, trimethoprim-sulfamethoxazole); the tetracyclines (e.g. demeclocycline, doxycycline, minocycline, oxytetracycline, tetracycline); the carbapenems (e.g. imipenem, meropenem, ertapenem, doripenem, panipenem/betamipron, biapenem, PZ-601); chloramphenicol; clindamycin, ethambutol; fosfomycin; isoniazid; linezolid; metronidazole; nitrofurantoin; pyrazinamide; quinupristin/dalfopristin; rifampin; spectinomycin; and vancomycin.

Representative antiseptics include, but are not limited to chlorine bleach (sodium hypochlorite), quaternary ammonium compounds (e.g. benzalkonium chloride, cetyl trimethylammonium bromide, cetylpyridinium chloride), hydrogen peroxide, phenol compounds (e.g. TCP Triclosan), alcohols (e.g. ethanol), Virkon™, iodine compounds (e.g. povidone-iodine), silver compounds (e.g. elemental silver nano/microparticles) and polybiguanides, including polyaminopropyl biguanide (PAPB) or polyhexamethylene biguanide (PHMB), chlorhexidine and alexidine.

Antimicrobial surfactants are a class of surfactant compounds that disrupt microbial cell membranes and other structural components and therefore inhibit growth and/or viability of microorganisms. Antimicrobial surfactants and their use in antimicrobial compositions is well known in the art should further guidance be needed the discussion of antimicrobial surfactants in “Preservative-free and self-preserving cosmetics and drugs—Principles and practice”, Ed. Kabara and Orth, Marcel Dekker, NY, N.Y., 1997, is explicitly incorporated by reference in its entirety. Antimicrobial surfactants may be anionic, cationic, non-ionic or amphoteric. Examples of antimicrobial anionic surfactants include, but are not limited to, sodium dodecyl sulfate (sodium lauryl sulfate), sodium dodecyl aminopropionic acid, sodium ricinoleate, bile acids, alkylaryl sulfonates, Grillosan DS7911, disodium undecylenic acid monoethanol amidosulfosuccinate. Examples of antimicrobial cationic surfactants include, but are not limited to, the quaternary ammionium compounds, the aminimides and chlorhexidine compounds. Examples of antimicrobial non-ionic surfactants include, but are not limited to, the monoesters of fatty acids, polyethyleneglycomonoesters of alkyldihydroxybenzoic acids, glucosamine derivatives and diethanolamides of N-lauroyl dipeptides. Examples of antimicrobial amphoteric surfactants include, but are not limited to, the alkyl betaines, the alkylamidopropylbetaines, the alkyl aminopropionates, the alkyliminodipropionates and the alkylimidazolines.

Representative antifungals include, but are not limited to the polyenes (e.g. natamycin, rimocidin, filipin, nystatin, amphotericin B, candicin); the imidazoles (e.g. miconazole, ketoconazole, clotrimazole, econazole, bifonazole, butoconazole, fenticonazole, isoconazole, oxiconazole, sertaconazole, sulconazole, tioconazole); the triazoles (e.g. fluconazole, itraconazole, isavuconazole, ravuconazole, posaconazole, voriconazole,terconazole); the allylamines (e.g. terbinafine, amorolfine, naftifine, butenafine); and the echinocandins (e.g. anidulafungin, caspofungin, micafungin).

Representative antivirals include, but are not limited to abacavir, acyclovir, adefovir, amantadine, amprenavir, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type, II interferon type I, lamivudine, lopinavir, loviride, maraviroc, moroxydine, nelfinavir, nevirapine, nexavir, oseltamivir, penciclovir, peramivir, pleconaril, podophyllotoxin, raltegravir, ribavirin, rimantadine, ritonavir, saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine

Of particular note are the antimicrobial peptides and the antimicrobial polyenes (e.g. a polyene antimycotic or polyene antibiotic), i.e. a polyene molecule that has antimicrobial activity, in particular antifungal/antimycotic activity, under appropriate conditions. Polyenes are poly-unsaturated organic compounds that contain one or more sequences of alternating double and single carbon-carbon bonds and would be immediate recognisable to the skilled man. Antimicrobial polyenes include, but are not limited to amphotericin B, nystatin, natamycin, rimocidin, filipin, hamycin, mepartricin and perimycin; amphotericin B, nystatin, natamycin being of note and natamycin most preferred.

An antimicrobial peptide is a polymer of up to 100, e.g. up to 90, 80, 70, 60, 50, 40, 30 or 20 amino acids linked by peptide bonds that has antimicrobial activity under appropriate conditions, preferably not involving the action of an immune system. The polymer may be linear or cyclic, or partially linear and partially cyclic, and may also be branched. The peptide bonds of the peptide need not all involve the a carbon of the constituent amino acids, and may for instance involve the side chain amine group of lysine or another amino acid side chains containing amine groups, produced by bacteria in the genus Streptomyces (e.g. Streptomyces albulus). Antimicrobial peptides typically have at least 5, e.g. at least 6, 7, 8, 9, 10, 11, 12, 13, 14, 15, 16, 17, 18 or 19 amino acids. Expressed differently an antimicrobial peptide may consist of about 5 to100 amino acids, e.g. about 6 to 90, 7 to 80, 8 to 70, 9 to 60, 10 to 50, 11 to 50, 12 to 50, 13 to 40, 15 to 35, 20 to 40, 25 to 35 or 25 to 30 amino acids.

Antimicrobial peptides have been isolated from a diverse range of sources and display a diverse range of structures, but these structures have been well characterised and documented and as such the skilled man would readily be able to identify or recognise an antimicrobial peptide or determine if a novel peptide is an antimicrobial peptide from the literature, his common general knowledge and routine experimental techniques. The following databases are a selection of those available to the skilled man as a resource of information on antimicrobial peptides: CAMP, APD, DAMPD, YADAMP, PhytAMP, RAPD, Defensins knowledgebase, AMPer, DADP, BACTIBASE, Peptaibol Database PenBase Penaeidin, Database, AntiBP2, BAGEL2, and LAMP. Antimicrobial peptides may be divided into subgroups based on their amino acid composition and structure. Nearly all antimicrobial peptides are cationic and very often amphiphilic. They include one or more positively charged residues (arginine, lysine or, in acidic environments, histidine), and a large proportion (>50%) of hydrophobic residues. Antimicrobial peptides can be roughly categorized into those that have a high content of a certain amino acid, most often proline, but also lysine, glutamine and arginine, those that contain intramolecular disulfide bridges, and those with an amphiphilic region in their molecule if they assume an α-helical structure. Secondary structures of antimicrobial peptides can be α-helical, β-stranded due to the presence of 2 or more disulfide bonds, β-hairpin or loop due to the presence of a single disulfide bond and/or cyclisation of the peptide chain, and extended.

Included in the term antimicrobial peptide are the peptide antibiotics, e.g. actinomycin, bacitracin, colistin, and polymyxin B; the glycopeptide antibiotics, e.g. teicoplanin, vancomycin, telavancin; and the lantibiotics, e.g. nisin (the lantibiotic produced by Lactococcus lactis), bisin, subtilin, epidermin, gallidermin, mutacin, mersacidin, actagardine, duramycin, cinnamycin, haloduracin, sublancin and plantaricin C.

Antimicrobial peptides having a net positive charge at or below physiological pH, e.g. a net charge of at least +1, e.g. at least +2, +3, +4, +5 or +10, are of note. This may also be expressed as an average positive charge per amino acid in the peptide at or below physiological pH of 0.1 to 3, e.g. 0.2 to 2, 0.3 to 1, 0.4 to 1, 0.5 to 1, 0.6 to 1, 0.7 to 1, 0.8 to 1, 0.9 to 1, or 1 to 3.

Preferred antimicrobial peptides include peptides (e.g. as defined above) of 10 to 30 amino acids, e.g. 10 to 25, 10 to 20, 10 to 15, 15 to 30, 15 to 20, 15 to 25, 20 to 30, 20 to 25 and 25 to 30 amino acids having a net positive charge (e.g. as defined above) consisting of (i) lysine, arginine, glutamine and/or histidine and (ii) alanine, glycine, leucine, isoleucine, valine, methionine, proline, phenylalanine and/or tryptophan. In preferred embodiments option (i) is lysine and/or arginine, or more preferably, lysine. In other preferred embodiments option (ii) is alanine, glycine, leucine, isoleucine and/or valine, or, more preferably, alanine, glycine and/or leucine. In certain embodiments, option (i) is 1, 2, 3, 4, 5, 6, 7, 8, 9 or 10 lysine residues, e.g. 1 to 6 lysine residues, with the remainder of the peptide consisting of option (ii) as defined above, e.g. alanine, glycine and/or leucine.

In accordance with the invention nisin, polyarginine and polyglutamine are preferred antimicrobial peptides.

In accordance with the invention antimicrobial peptides that have activity against bacteria and/or fungi are preferred.

In these embodiments it may be advantageous to select antimicrobial agents that have different target microorganisms, thereby expanding/tailoring the antimicrobial spectrum of the antimicrobial preparation.

The aqueous antimicrobial composition may comprise suitable carriers, excipients, and diluents, e.g. to enhance storage life, sprayability, surface adhesion and surface retention, to meet safety requirements (e.g. dyes and bittering agents), to improve flavour, sweetness and smell or to control the physical properties of the formulation. Examples of suitable compounds are glucose, sucrose, maltose, galactose, fructose, lactose, dextrose, sucrose, sorbitol, mannitol, starches, gum acacia, calcium phosphate, inert alginates, tragacanth, gelatine, calcium silicate, microcrystalline cellulose, polyvinylpyrrolidone, cellulose, glycol, polyethylene, propylene glycol, methyl cellulose, methylhydroxybenzoates, propyl hydroxybenzoates, talc, magnesium stearate, pH buffers, alkalis and inorganic acids.

Compounds which may enhance the surface adherence and/or retention of the antimicrobial components of the aqueous antimicrobial composition are well known to the skilled man although particular examples include polyarginine, polyglutamine, polylysine (e.g. E polylysine; referred to herein as PPL), abaecin, bactenecin, lactoferricin B, cecropin C. ε-polylysine (PPL) is a peptide produced by bacteria in the genus Streptomyces (e.g. Streptomyces albulus) in which the side chain amine group of lysine forms peptide bonds with other lysine residues. PPL typically has 20 to 45, 20 to 40, 25 to 35 or 25 to 30 amino acids joined in this way.

By way of example the PPL (or other surface adherence agents) may be included in the antimicrobial composition at a concentration of about 50 μM to about 10 mM.

To the extent constrained by the above recited range endpoints, the PPL (or other surface adherence agents) may be included in the antimicrobial composition at a concentration of about 50 μM or greater, e.g. about 100 μM, 150 μM, 200 μM, 250 μM, 300 μM, 350 μM, 400 μM, 450 μM, 500 μM, 550 μM, 600 μM, 650 μM, 700 μM, 750 μM, 800 μM, 850 μM, 900 μM, 950 μM, 1 mM, 2 mM, 3 mM, 4 mM, 5 mM, 6 mM, 7 mM, 8 mM or 9 mM or greater.

To the extent constrained by the above recited range endpoints, in other embodiments the PPL (or other surface adherence agents) may be included in the compositions of the invention at a concentration of equal to or less than about 10 mM, e.g. equal to or less than about 9 mM, 8 mM, 7 mM, 6 mM, 5 mM, 4 mM, 3 mM, 2 mM, 1 mM, 950 μM, 900 μM, 850 μM, 800 μM, 750 μM, 700 μM, 650 μM, 600 μM, 550 μM, 500 μM, 450 μM, 400 μM, 350 μM, 300 μM, 250 μM, 200 μM, 150 μM or 100 μM.

Ranges of concentrations with endpoints formed from the combination of any and all of the concentrations recited above are explicitly contemplated. By way of example the PPL (or other surface adherence agents) may be included in the compositions of the invention at a concentration of about 50 μM to about 10 mM, e.g. about 100 μM, 150 μM, 200 μM, 250 μM, 300 μM, 350 μM, 400 μM, 450 μM, 500 μM, 550 μM, 600 μM, 650 μM, 700 μM, 750 μM, 800 μM, 850 μM, 900 μM, 950 μM, 1 mM, 2 mM, 3 mM, 4 mM, 5 mM, 6 mM, 7 mM, 8 mM or 9 mM to about 10 mM.

In another example the PPL (or other surface adherence agents) may be included in the compositions of the invention at a concentration of about 50 μM to about 9 mM, e.g. about 50 μM to about 8 mM, 7 mM, 6 mM, 5 mM, 4 mM, 3 mM, 2 mM, 1 mM, 950 μM, 900 μM, 850 μM, 800 μM, 750 μM, 700 μM, 650 μM, 600 μM, 550 μM, 500 μM, 450 μM, 400 μM, 350 μM, 300 μM, 250 μM, 200 μM, 150 μM or 100 μM.

These compositions in particular may be considered ready to use compositions.

Thus, in further embodiments the antimicrobial aqueous composition may comprise:
(i) a decylglucoside at a concentration of about 2 mM to about 40 mM,
(ii) at least one C₁ to C₈ carboxylic acid, or a water-soluble salt thereof, at a concentration of about 1 μM to about 100 mM, and
(iii) PPL at a concentration of about 50 μM to about 10 mM.

In further preferred embodiments the antimicrobial aqueous composition consists substantially, e.g. essentially, of a decylglucoside at a concentration of about 2 mM to about 40 mM, at least one C₁ to C₈ carboxylic acid, or a water-soluble salt thereof, at a concentration of about 1 μM to about 100 mM, PPL at a concentration of about 50 μM to about 10 mM and water, i.e. any excipients, carriers or other active agents are present in negligible amounts. This may be expressed as an antimicrobial aqueous composition consisting of the following:
(i) a decylglucoside at a concentration of about 2 mM to about 40 mM,
(ii) at least one C₁ to C₈ carboxylic acid, or a water-soluble salt thereof, at a concentration of about 1 μM to about 100 mM,
(iii) PPL at a concentration of about 50 μM to about 10 mM,
(iv) equal or less than about 10% w/w (e.g. equal or less than about 9%, 8%, 7%, 6%, 5%, 4%, 3%, 2%, 1%, 0.5%, 0.1%, or 0.01% w/w) in total of excipients, carriers or active agents other than water, and
(v) an amount of water sufficient to provide a total percentage w/w of 100%.

In other embodiments the antimicrobial aqueous composition consists of water and the following dissolved therein:
(i) a decylglucoside at a concentration of about 2 mM to about 40 mM,
(ii) at least one C₁ to C₈ carboxylic acid, or a water-soluble salt thereof, ata concentration of about 1 μM to about 100 mM, and
(iii) PPL at a concentration of about 50 μM to about 10 mM.

In these embodiments it is preferred that the at least one C₁ to C₈ carboxylic acid, or water soluble salt thereof, is lactic acid, or water soluble salt thereof, and/or citric acid, or water soluble salt thereof.

In other embodiments the antimicrobial aqueous composition consists of a decylglucoside, lactic acid, or water soluble salt thereof, PPL and water, wherein:
(i) the decylglucoside is present a concentration of about 300 ppm to about 3000 ppm,
(ii) the lactic acid, or water soluble salt thereof, is present at a concentration of about 0.1 ppm to about 10000 ppm, and
(iii) the PPL is present at a concentration of about 10 ppm to about 2000 ppm.

To the extent constrained by the above recited range endpoints, in these embodiments the PPL may be present at a concentration of about 10 ppm or greater, e.g. about 20 ppm, 30 ppm, 40 ppm, 50 ppm, 60 ppm, 70 ppm, 80 ppm, 90 ppm, 100 ppm, 150 ppm, 200 ppm, 250 ppm, 300 ppm, 350 ppm, 400 ppm, 450 ppm, 500 ppm, 550 ppm, 600 ppm, 650 ppm, 700 ppm, 750 ppm, 800 ppm, 850 ppm, 900 ppm, 950 ppm, 1000 ppm, 1100 ppm, 1200 ppm 1300 ppm, 1400 ppm, 1500 ppm, 1600 ppm, 1700 ppm, 1800 ppm or 1900 ppm or greater.

To the extent constrained by the above recited range endpoints, in other embodiments the PPL may be present at a concentration of equal to or less than about 2000 ppm, e.g. equal to or less than about 1900 ppm, 1800 ppm, 1700 ppm, 1600 ppm, 1500 ppm, 1400 ppm, 1300 ppm, 1200 ppm, 1100 ppm, 1000 ppm, 950 ppm, 900 ppm, 850 ppm, 800 ppm, 750 ppm, 700 ppm, 650 ppm, 600 ppm, 550 ppm, 500 ppm, 450 ppm, 400 ppm, 350 ppm, 300 ppm, 250 ppm, 200 ppm, 150 ppm, 100 ppm, 90 ppm, 80 ppm, 70 ppm, 60 ppm, 50 ppm, 40 ppm, 30 ppm, or 20 ppm.

Ranges of concentrations with endpoints formed from the combination of any and all of the concentrations recited above are explicitly contemplated. By way of example the PPL may be present at a concentration of about 10 ppm to about 2000 ppm, e.g. about 20 ppm, 30 ppm, 40 ppm, 50 ppm, 60 ppm, 70 ppm, 80 ppm, 90 ppm, 100 ppm, 150 ppm, 200 ppm, 250 ppm, 300 ppm, 350 ppm, 400 ppm, 450 ppm, 500 ppm, 550 ppm, 600 ppm, 650 ppm, 700 ppm, 750 ppm, 800 ppm, 850 ppm, 900 ppm, 950 ppm, 1000 ppm, 1100 ppm, 1200 ppm 1300 ppm, 1400 ppm, 1500 ppm, 1600 ppm, 1700 ppm, 1800 ppm, 1900 ppm to about 2000 ppm.

In another example the PPL may be present at a concentration of about 10 ppm to about 1900 ppm, e.g. about 10 ppm to about 1800 ppm, 1700 ppm, 1600 ppm, 1500 ppm, 1400 ppm, 1300 ppm, 1200 ppm, 1100 ppm, 1000 ppm, 950 ppm, 900 ppm, 850 ppm, 800 ppm, 750 ppm, 700 ppm, 650 ppm, 600 ppm, 550 ppm, 500 ppm, 450 ppm, 400 ppm, 350 ppm, 300 ppm, 250 ppm, 200 ppm, 150 ppm, 100 ppm, 90 ppm, 80 ppm, 70 ppm, 60 ppm, 50 ppm, 40 ppm, 30 ppm, or 20 ppm.

These compositions in particular may be considered ready to use compositions.

The above recited ppm values for the concentrations of decylglucoside and lactic acid, or water soluble salt thereof, apply mutatis mutandis.

In other embodiments the invention provides an antimicrobial aqueous composition consisting of a decylglucoside, citric acid, or water soluble salt thereof, PPL and water, wherein:
(i) the decylglucoside is present at a concentration of about 300 ppm to about 3000 ppm,
(ii) the citric acid, or water soluble salt thereof, is present at a concentration of about 0.2 ppm to about 20000 ppm, and
(iii) the PPL is present at a concentration of about 10 ppm to about 2000 ppm.

The above recited ppm values for the concentrations of PPL, decylglucoside and citric acid apply mutatis mutandis.

In other embodiments the invention provides an antimicrobial aqueous composition consisting of a decylglucoside, citric acid, or water soluble salt thereof, lactic acid, or water soluble salt thereof, PPL and water wherein:
(i) the decylglucoside is present at a concentration of about 300 ppm to about 3000 ppm,
(ii) the lactic acid, or water soluble salt thereof, is present at a concentration of about 0.05 ppm to about 5000 ppm,
(iii) the citric acid, or water soluble salt thereof, is present at a concentration of about 0.1 ppm to about 10000 ppm, and
(iv) the PPL is present at a concentration of at least about 10 ppm to about 2000 ppm.

The above recited ppm values for the concentrations of PPL, decylglucoside, lactic acid, or water soluble salt thereof, and citric acid, or water soluble salt thereof, apply mutatis mutandis.

The specific compositions containing octylglucoside and decylglucoside described above may also include PPL and the specific features of the above disclosure of PPL-containing and decylglucoside-containing compositions of the invention apply mutatis mutandis to those specific compositions containing octylglucoside and decylglucoside.

In particular embodiments the invention provides a (ready to use) antimicrobial aqueous composition consisting of a decylglucoside, an octylglucoside, lactic acid, or water soluble salt thereof, PPL and water wherein:
(i) the decylglucoside is present at a concentration of about 600 ppm
(ii) the octylglucoside is present at a concentration of about 400 ppm
(iii) the lactic acid, or water soluble salt thereof, is present at a concentration of about 200 ppm (2 mM), and
(iv) the PPL is present at a concentration of about 100 ppm.

References to PPL in the above recited embodiments may be taken to extend to other surface adherence agents as appropriate.

The aqueous antimicrobial compositions of the invention are preferably liquid at the temperatures at which they are used, e.g. are liquid at temperatures above 0° C. when under 1 atm pressure. Carriers that influence the viscosity and/or rheology of the composition are also of particular note as further components of the compositions of the invention. More specifically the inclusion of polymer carriers in the composition may allow for the composition to be provided in a form other than a simple liquid, e.g. as a viscous liquid, a semi solid or a solid, e.g. as a hydrogel or hydrocolloid gel. Such forms may be advantageous in the sanitisation of body surfaces, e.g. skin, scalp, hair, teeth, gums and other oral, buccal or dental surfaces. In this context toothpastes, dental gels, dental foams, oral sprays and mouthwashes are mentioned specifically. Gel-forming (e.g. hydrogel or hydrocolloid gel forming) polymeric substances, include, but are not limited to, alginate, cellulose (e.g. oxidised regenerated cellulose, methylcellulose, carboxymethylcellulose, hydroxyethylcellulose), collagen, pectin, elastin, fibronectin.

The aqueous compositions of the invention preferably do not comprise a bactericidal alcohol. Bactericidally active alcohols include aliphatic alcohols and phenyl-aliphatic alcohols, i.e. aliphatic alcohols substituted by phenyl groups in the aliphatic C-chain, which may contain one or more hydroxyl groups. Of the aliphatic alcohols, straight-chain or branched, unsubstituted or mono- or disubstituted aliphatic alcohols containing from 1 to 6 carbon atoms in the alkyl or alkylene radical are mentioned specifically. Within the above-mentioned group, straight-chain or branched unsubstituted aliphatic alcohols containing from 2 to 4 carbon atoms in the alkyl or alkylene radical are mentioned specifically. Alcohols such as these are ethanol, n-propanol or isopropanol. The mono- or disubstituted aliphatic alcohols mentioned above may be straight-chain or branched, aliphatic C₂-C₄ alcohols substituted by 1 or 2 substituents from the group Cl, Br or NO₂. Of these compounds, 2-bromo-2-nitro-1,3-propane diol is mentioned specifically. Straight-chain or branched, unsubstituted or mono- or disubstituted phenyl-aliphatic alcohols containing from 1 to 3 carbon atoms in the alkylene radical instead of straight aliphatic alcohols are mentioned specifically. “Phenyl-aliphatic” alcohols are understood to be alcohols in which the alcohol function is attached to the alkyl chain and the alkyl radical additionally contains a phenyl radical as substituent. Within the group of alcohols such as these, straight-chain, unsubstituted phenylaliphatic alcohols containing 1 to 3 carbon atoms in the alkylene radical or straight-chain phenyl-aliphatic alcohols containing from 1 to 3 carbon atoms in the alkylene radical substituted by 1 or 2 substituents from the group comprising Cl, Br or NO₂, e.g. benzyl-alcohol, are mentioned specifically.

Where the use to which the antimicrobial composition is put is in the in vitro treatment of inanimate surfaces and locations the composition may be considered a sanitising, disinfecting, antiseptic or sterilising formulation.

Where the use to which the antimicrobial composition is put is in the combat of microbial contamination on an animate surface, i.e. a surface of and on or in a living human or animal body (in vivo), or an inanimate surface on or in a living human or animal body, in other words as a pharmaceutical, the aqueous composition is provided as a pharmaceutically acceptable composition or formulation for application to the surface to be treated. Such formulations can also be used in ex vivo treatments of tissues and body parts. The skilled person would be familiar with suitable pharmaceutically acceptable carriers, excipients and diluents and would understand which of those recited above are in such categories.

Thus, in a further embodiment the antimicrobial aqueous composition of the invention is provided as an antimicrobial aqueous pharmaceutical composition, i.e. comprising the recited components and, if present, pharmaceutically acceptable carriers, diluents or excipients.

Where the use to which the antimicrobial aqueous composition us put is in the combat of microbial contamination on an inanimate surface in vitro, i.e. not on or in a living human or animal body, the antimicrobial aqueous composition may be applied to the surface to be treated by any convenient means. For instance a liquid form of the antimicrobial aqueous composition of the invention may simply be sprayed onto the inanimate surface to be treated, e.g. via a misting system or via a spray gun or other atomising device which may be manual, semi-automated or fully automated. Spraying may involve a propellant but preferably will be propellant free. Spray applications may involve a pre- and/or post application spraying of water or other aqueous solution. In other examples a liquid or gel form of the antimicrobial aqueous composition of the invention may be applied, e.g. via a fabric or sponge wipe carrying the composition.

A typical misting system/infrastructure (which terms are used interchangeably) of use in accordance with the invention may comprise a spray nozzle (also referred to as an atomiser or a nebuliser) to which the ready to use aqueous liquid composition of use in the invention is delivered at sufficient pressure to atomise the liquid thereby producing a spray (mist) of suitable droplet size to effect successful administration/application to the treatment site and a remote reservoir of the liquid to be sprayed. Delivery of the liquid to the nozzle is typically via conduits (e.g. pipes, hosing, tubing or waterlines). In other embodiments a reservoir of a concentrated liquid form or dry solid form of the aqueous liquid composition of the invention is provided and dilution of that composition with a suitable aqueous diluent, e.g. water, is effected in a separate reservoir or in the conduit between the reservoir of a concentrated aqueous liquid composition and the spray nozzle. Thus, a preferred misting system of the invention may comprise a nozzle, a reservoir adapted to contain a concentrated aqueous liquid composition of the invention, a conduit between the reservoir and the nozzle and means for delivering a suitable aqueous solvent to the concentrated aqueous liquid composition prior to its expulsion through the nozzle. The misting system may have a further reservoir adapted to contain the aqueous solvent. The misting system may be integrated within a building or structure or be free-standing.

The misting system may be manual, semi-automated or automated. The misting system may therefore be controlled by a computer program and thus a further component of a misting system of use in the invention may be a computer, system or apparatus carrying a program adapted to control the misting system, preferably adapted to perform an automated or semi-automated misting protocol.

Thus in a further aspect the invention provides a misting system, specifically a residual misting system (i.e. a system that provides a spray that leaves a residue on a treatment site) containing one or more spray nozzles, a first reservoir and a conduit between the first reservoir and the spray nozzles adapted to convey the contents of the first reservoir to the spray nozzle, wherein the misting system, or portion thereof (e.g. said first reservoir) contains an antimicrobial aqueous composition of the invention as defined herein. In preferred embodiments the misting system further comprises the means to deliver an aqueous diluent to the first reservoir, a mixing receptacle, the conduit or the nozzle thereby diluting at least a portion of the contents of the first reservoir. In further embodiments the misting system further comprises a second reservoir containing a suitable aqueous solvent. In still further embodiments the misting system further comprises a computer, system or apparatus carrying a program adapted to control the misting system, preferably adapted to perform an automated or semi-automated misting protocol.

“Antimicrobial” in accordance with the invention means the ability of an agent to kill, destroy, or inhibit the growth of, microorganisms. This may therefore be a microbicidal activity and/or a microbiostatic activity.

More particularly, the term “microbicidal” means the ability negatively to impact the viability (i.e. to reduce or inhibit or ablate the viability) of a microorganism. In particular, “microbicidal” means the ability to kill or destroy a microorganism. The terms “kill” and “destroy” encompass the complete or partial destruction of the microorganism, e.g. the full or partial disintegration of the cellular structure of a microorganism. “Microbiostatic” means the ability to inhibit the growth of a microorganism. As described further below, the term “growth” is used broadly herein to refer to any aspect of growth of a microorganism, including both an increase in size or in the numbers of a microorganism. The term “growth” thus explicitly includes replication or reproduction of a microorganism. The term “inhibit” includes any degree of reduction of growth (as compared for example to growth which may be observed in the absence of the microbiostatic agent) as well prevention of growth.

The term “microbicidal” thus includes a cytotoxic effect of an agent against a microorganism. Therefore, a microbicidal agent can be viewed as bactericidal, fungicidal, algicidal, protozoacidal and so on depending on the type of microorganism that the agent is cytotoxic against. Similarly, the term “microbiostatic” can be viewed as a reference to a cytostatic effect of an agent against a microorganism. Therefore, in relation to the term “microbe” a microbiostatic agent can be categorised as bacteriostatic, fungistatic, algistatic, protozoastatic and so on depending on the type of microbe that the agent is cytostatic against.

The term “viability of a microorganism” means the ability of a microbe to survive under given conditions. Survival can be considered equivalent to remaining alive. Determining the viability of a microorganism can be done using the techniques detailed below for measuring microorganism cell death (and viability).

The term “killing a microorganism” refers to the act of causing a microorganism to cease to be alive, i.e. to become dead. A microorganism is considered to be alive if it can be induced to replicate and/or grow, or at least display morphological changes, when placed in a medium that would normally support the growth of that microorganism and/or the microorganism is metabolising nutrients to release energy to support cellular functions. Typically, a microorganism can be considered to be dead if cell membrane integrity is lost.

Many routine assays are available to determine if a microorganism is alive (viable) or dead. One option is to place the microorganism in conditions that would normally support the growth of that microorganism and monitor the growth of the microorganism by appropriate standard means, e.g. by monitoring the size of the microorganism, the morphology of the microorganism, the number of microorganisms in the colony over time, the consumption of nutrients in the culture media, etc. Another option is to assess the microorganism for morphologies characteristic of cell death, e.g. necrotic or apoptotic bodies, membrane blebs, nuclear condensation and cleavage of DNA into regularly sized fragments, ruptured cell walls or membranes and leakage of cell contents into the extracellular environment. Other methods exploit the characteristic loss of cell membrane integrity in dead microorganisms. Membrane impermeable dyes (e.g. trypan blue and propidium iodide) are routinely used to assess membrane integrity. A still further option is to measure the metabolism of the microorganism. This can be done routinely in a number of ways. For instance the levels of ATP can be measured

By “growth of a microorganism” it is meant both an increase in the size of the microorganism or in the amount and/or volume of the constituents of a microorganism (e.g. the amount of nucleic acid, the amount of protein, the number of nuclei, the numbers or size of organelles, the volume of cytoplasm) and an increase in the numbers of a microorganism i.e. an increase in the replication of a microorganism.

By “inhibiting the growth of a microorganism” it is meant that measurable growth (e.g. replication) of a microorganism, or the rate thereof, is reduced. Preferably measurable growth (e.g. replication) of a microorganism, or the rate thereof, is reduced by at least 50%, more preferably at least 60%, 70%, 80% or 90%, e.g. at least 95%. Preferably, measurable growth (e.g. replication) is ceased. Growth in terms of microbial size increase or expansion etc. may be inhibited independently of replication and vice versa

The subject may be any human or non-human animal subject, but more particularly may be a vertebrate, e.g. an animal selected from mammals, birds, amphibians, fish and reptiles. The animal may be a livestock or a domestic animal or an animal of commercial value, including laboratory animals or an animal in a zoo or game park. Representative animals therefore include dogs, cats, rabbits, mice, guinea pigs, hamsters, horses, pigs, sheep, goats, cows, chickens, turkeys, guinea fowl, ducks, geese, parrots, budgerigars, pigeons, salmon, trout, cod, haddock, sea bass and carp. Veterinary uses of the invention are thus covered. The subject may be viewed as a patient. Preferably the subject is a human.

The term “in a subject” is used broadly herein to include sites or locations inside a subject or on a subject, e.g. an external body surface, and may include in particular infection of a medical device e.g. an implanted or “in-dwelling” medical device.

The subject may be, suspected to be, or at risk of infection. The methods of the invention may comprise a preceding step in which the subject is determined to be, or at risk of infection. The methods may have a following step in which the subject's clinical indicators of infection are assessed and optionally compared to a similar assessment from earlier in their treatment or from before treatment began.

“Treatment” when used in relation to the treatment of a medical condition/infection in a subject in accordance with the invention is used broadly herein to include any therapeutic effect, i.e. any beneficial effect on the condition or in relation to the infection. Thus, not only included is eradication or elimination of the infection, or cure of the subject or infection, but also an improvement in the infection or condition of the subject. Thus included for example, is an improvement in any symptom or sign of the infection or condition, or in any clinically accepted indicator of the infection/condition (for example a decrease in body temperature and/or markers or signs of inflammation). Treatment thus includes both curative and palliative therapy, e.g. of a pre-existing or diagnosed infection/condition, i.e. a reactionary treatment.

“Prevention” as used herein refers to any prophylactic or preventative effect. It thus includes delaying, limiting, reducing or preventing the condition (which reference includes infection, colonisation and contamination, as applicable, in the different aspects of the invention) or the onset of the condition, or one or more symptoms or indications thereof, for example relative to the condition or symptom or indication prior to the prophylactic treatment. Prophylaxis thus explicitly includes both absolute prevention of occurrence or development of the condition, or symptom or indication thereof, and any delay in the onset or development of the condition or symptom or indication, or reduction or limitation on the development or progression of the condition or symptom or indication.

In a further aspect the invention provides an antimicrobial aqueous composition as defined above, i.e. an antimicrobial aqueous composition comprising:
(i) a decylglucoside at a concentration of about 2 mM to about 40 mM, and
(ii) at least one C₁ to C₈ carboxylic acid, or a water-soluble salt thereof, at a concentration of about 1 μM to about 100 mM.

All preferred embodiments of such a composition recited above apply mutated mutandis to this aspect of the invention. Such compositions may be considered to be compositions which are “ready to use” in the methods and uses of the first aspect of the invention.

Advantageously, the ready to use forms of the aqueous antimicrobial compositions of the invention may be provided in a concentrated form which, in turn, may be diluted with an aqueous diluent to form the ready to use compositions.

Therefore in another aspect the invention provides an antimicrobial aqueous composition consisting substantially, e.g. essentially, of
(i) a decylglucoside at a concentration of about 2 mM or greater,
(ii) at least one C₁ to C₈ carboxylic acid, or a water-soluble salt thereof, at a concentration of about 1 μM or greater, optionally
(iii) an octylglucoside at a concentration of about 0.2 mM (about 30 ppm) or greater, preferably wherein the ratio of said octylglucoside to said decylglucoside is at least about 10:100, optionally
(iv) an surface adherence agent at a concentration of about 50 μM or greater, and
(v) water

In these embodiments any other excipients, carriers or other active agents, if present, are present in negligible amounts.

This may be expressed as an antimicrobial aqueous composition consisting of the following:
(i) a decylglucoside at a concentration of about 2 mM or greater,
(ii) at least one C₁ to C₈ carboxylic acid, or a water-soluble salt thereof, at a concentration of about 1 μM or greater, optionally
(iii) an octylglucoside at a concentration of about 0.2 mM (about 30 ppm) or greater, preferably wherein the ratio of said octylglucoside to said decylglucoside is at least about 10:100, optionally
(iv) an surface adherence agent at a concentration of about 50 μM or greater,
(v) equal or less than about 0.5% w/w (e.g. equal or less than about 0.1%, or 0.01% w/w) in total of excipients, carriers or active agents other than water, and
(vi) an amount of water sufficient to provide a total percentage w/w of 100%.

In other specific embodiments the invention provides an antimicrobial aqueous composition consisting of water and the following dissolved therein:
(i) a decylglucoside at a concentration of about 2 mM or greater,
(ii) at least one C₁ to C₈ carboxylic acid, or a water-soluble salt thereof, at a concentration of about 1 μM or greater.

In other specific embodiments the invention provides an antimicrobial aqueous composition consisting of water and the following dissolved therein:
(i) a decylglucoside at a concentration of about 2 mM or greater,
(ii) an octylglucoside at a concentration of about 0.2 mM or greater, preferably wherein the ratio of said octylglucoside to said decylglucoside is at least 10:100, and
(iii) at least one C₁ to C₈ carboxylic acid, or a water-soluble salt thereof, at a concentration of about 1 μM or greater.

In other specific embodiments the invention provides an antimicrobial aqueous composition consisting of water and the following dissolved therein:
(i) a decylglucoside at a concentration of about 2 mM or greater,
(ii) at least one C₁ to C₈ carboxylic acid, or a water-soluble salt thereof, at a concentration of about 1 μM or greater, and
(iii) a surface adherence agent (e.g. PPL) at a concentration of about 50 μM or greater.

In other embodiments the invention provides an antimicrobial aqueous composition consisting of water and the following dissolved therein:
(i) a decylglucoside at a concentration of about 2 mM or greater,
(ii) at least one C₁ to C₈ carboxylic acid, or a water-soluble salt thereof, at a concentration of about 1 μM or greater,
(iii) an octylglucoside at a concentration of about 0.2 mM or greater, preferably wherein the ratio of said octylglucoside to said decylglucoside is at least 10:100, and
(iv) a surface adherence agent (e.g. PPL) at a concentration of about 50 μM or greater.

In this aspect the composition may comprise the decylglucoside at a concentration of about 2.1 mM or greater, e.g. about 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.5 mM, 5 mM, 6 mM, 7 mM, 8 mM, 9 mM, 10 mM, 12 mM, 14 mM, 16 mM, 18 mM, 20 mM, 30 mM, 40 mM, 50 mM, 60 mM, 80 mM, 100 mM, 150 mM, 200 mM, or greater. This may be expressed as a concentration of about 300 ppm or greater, e.g. about 310 ppm, 320 ppm, 330 ppm, 340 ppm, 350 ppm, 360 ppm, 370 ppm, 380 ppm, 390 ppm, 400 ppm, 410 ppm, 420 ppm, 430 ppm, 440 ppm, 450 ppm, 460 ppm, 470 ppm, 480 ppm, 490 ppm, 500 ppm, 550 ppm, 600 ppm, 700 ppm, 800 ppm, 900 ppm, 1000 ppm, 1100 ppm, 1200 ppm, 1400 ppm, 1600 ppm, 1800 ppm, 2000 ppm, 2500 ppm or 3000 ppm or greater.

In this aspect the composition may comprise the at least one C₁ to C₈ carboxylic acid, or a water-soluble salt thereof, at a concentration of about 5 μM or greater, e.g. 10 μM, 25 μM, 50 μM, 100 μM, 200 μM, 250 μM, 500 μM, 800 μM, 1 mM, 1.6 mM, 2 mM, 2.4 mM, 2.5 mM, 3 mM, 4 mM, 5 mM, 5.6 mM, 6 mM, 7 mM, 8 mM, 9 mM, 10 mM, 20 mM, 30 mM, 40 mM, 50 mM, 60 mM, 70 mM, 80 mM, 90 mM or 100 mM or greater.

In these embodiments it is preferred that the at least one C₁ to C₈ carboxylic acid, or water soluble salt thereof, is lactic acid, or water soluble salt thereof, and/or citric acid, or water soluble salt thereof.

If selected, lactic acid, or water soluble salt thereof, may be present at a concentration of about 0.1 ppm or greater, e.g. 0.5 ppm, 1 ppm, 2.5 ppm, 5 ppm, 10 ppm, 20 ppm, 25 ppm, 50 ppm, 80 ppm, 100 ppm, 160 ppm, 200 ppm, 240 ppm, 250 ppm, 300 ppm, 400 ppm, 500 ppm, 560 ppm, 600 ppm, 700 ppm, 800 ppm, 900 ppm, 1000 ppm, 2000 ppm, 3000 ppm, 4000 ppm, 5000 ppm, 6000 ppm, 7000 ppm, 8000 ppm, 9000 ppm or 10000 ppm or greater. If selected citric acid, or water soluble salt thereof, may be present at a concentration of about 0.2 ppm or greater, e.g. 0.5 ppm, 1 ppm, 2.5 ppm, 5 ppm, 10 ppm, 20 ppm, 25 ppm, 50 ppm, 80 ppm, 100 ppm, 160 ppm, 200 ppm, 240 ppm, 250 ppm, 300 ppm, 400 ppm, 500 ppm, 560 ppm, 600 ppm, 700 ppm, 800 ppm, 900 ppm, 1000 ppm, 2000 ppm, 3000 ppm, 4000 ppm, 5000 ppm, 6000 ppm, 7000 ppm, 8000 ppm, 9000 ppm, 10000 ppm, 15000 ppm or 20000 ppm or greater. If both lactic acid, or water soluble salt thereof, and/or citric acid, or water soluble salt thereof are selected specific ppm values for the concentration of lactic acid, or water soluble salt thereof, and citric acid, or water soluble salt thereof may be derived from those recited above by halving said respective values

In this aspect the composition may comprise the octylglucoside at a concentration of about 0.21 mM or greater, e.g. about 0.22 mM, 0.23 mM, 0.24 mM, 0.25 mM, 0.26 mM, 0.27 mM, 0.28 mM, 0.29 mM, 0.3 mM, 0.31 mM, 0.32 mM, 0.33 mM, 0.34 mM, 0.35 mM, 0.36 mM, 0.37 mM, 0.38 mM, 0.39 mM, 0.4 mM, 0.45 mM, 0.5 mM, 0.6 mM, 0.7 mM, 0.8 mM, 0.9 mM, 1 mM, 1.2 mM, 1.4 mM, 1.6 mM, 1.8 mM, 2 mM, 3 mM, 4 mM, 4.5 mM, 5 mM, 6 mM, 7 mM, 8 mM, 9 mM, 10 mM, 12 mM, 14 mM, 16 mM, 18 mM, 20 mM, 30 mM, 40 mM, 50 mM, 60 mM, 80 mM, 100 mM, 150 mM, 200 mM, or greater. This may be expressed as a concentration of about 30 ppm or greater, e.g. about 31 ppm, 32 ppm, 33 ppm, 34 ppm, 35 ppm, 36 ppm, 37 ppm, 38 ppm, 39 ppm, 40 ppm, 41 ppm, 42 ppm, 43 ppm, 44 ppm, 45 ppm, 46 ppm, 47 ppm, 48 ppm, 49 ppm, 50 ppm, 55 ppm, 60 ppm, 70 ppm, 80 ppm, 90 ppm, 100 ppm, 110 ppm, 120 ppm, 140 ppm, 160 ppm, 180 ppm, 200 ppm, 250 ppm, 300 ppm, 400ppm, 450 ppm, 600 ppm, 750 ppm, 900 ppm, 1050 ppm, 1200 ppm, 1350 ppm, 1500 ppm, 1800 ppm, 2100 ppm, 2400 ppm, 2700 ppm or 3000 ppm or greater.

In this aspect the composition may comprise a surface adherence agent (e.g. PPL) at a concentration of about 100 μM or greater, e.g. about 150 μM, 200 μM, 250 μM, 300 μM, 350 μM, 400 μM, 450 μM, 500 μM, 550 μM, 600 μM, 650 μM, 700 μM, 750 μM, 800 μM, 850 μM, 900 μM, 950 μM, 1 mM, 2 mM, 3 mM, 4 mM, 5 mM, 6 mM, 7 mM, 8 mM, 9 mM, 10 mM, or greater. This may be expressed as a concentration of about 10 ppm or greater, e.g. about 20 ppm, 30 ppm, 40 ppm, 50 ppm, 60 ppm, 70 ppm, 80 ppm, 90 ppm, 100 ppm, 150 ppm, 200 ppm, 250 ppm, 300 ppm, 350 ppm, 400 ppm, 450 ppm, 500 ppm, 550 ppm, 600 ppm, 650 ppm, 700 ppm, 750 ppm, 800 ppm, 850 ppm, 900 ppm, 950 ppm, 1000 ppm, 1100 ppm, 1200 ppm 1300 ppm, 1400 ppm, 1500 ppm, 1600 ppm, 1700 ppm, 1800 ppm, 1900 ppm or 2000 ppm or greater.

In this aspect, to the extent the compositions may contain negligible amounts of excipients, carriers or active agents other than water, those excipients may be any of those described herein and any and all preferred features relating to those excipients, carriers or active agents other than water may apply mutatis mutandis.

In a further aspect the invention provides a dry solid composition comprising amounts of a decylglucoside and at least one C₁ to C₈ carboxylic acid, or a water-soluble salt thereof, that following contact with a sufficient amount of an aqueous liquid, e.g. water, provides a ready to use form of the antimicrobial aqueous composition as described herein, i.e. an antimicrobial aqueous composition comprising:

(i) a decylglucoside at a concentration of about 2 mM to about 40 mM, and

(ii) at least one C₁ to C₈ carboxylic acid, or a water-soluble salt thereof, at a concentration of about 1 μM to about 100 mM.

The above discussion of the features of the antimicrobial aqueous compositions of use in the antimicrobial methods of the invention applies mutatis mutandis as appropriate to this aspect. In particular the dry solid composition may consist of the components of the concentrated compositions of the invention described herein.

By “dry” it is meant that the solid composition is substantially, e.g. essentially, water-free (moisture-free). This may be expressed as a water content of less than 5% w/w, e.g. less than 4.5%, 4%, 3.5%, 3%, 2.5%, 2%, 1.5% or 1% w/w as measured by weight loss on drying or chemically by the Karl Fischer method (United States Pharmacopeia; European Pharmacopoeia). The solid composition of the invention is preferably dried by freeze drying (lyophilisation).

The precise form of the solid composition of the invention is not limited. It may be single entity or in finely divided, e.g. particulate or powder, form. In certain embodiments the powder/particulate form may be provided compressed into a tablet or encapsulated. The solid may be a dry gel or sponge or a film or sheet.

In a further aspect the invention provides a kit comprising, optionally in separate containers:
(i) a decylglucoside, optionally in an aqueous solution;
(ii) at least one C₁ to C₈ carboxylic acid, or a water-soluble salt thereof, optionally in an aqueous solution; and
(iii) instructions to prepare an antimicrobial aqueous composition comprising said decylglucoside at a concentration of about 2 mM to about 40 mM and said at least one C₁ to C₈ carboxylic acid, or water-soluble salt thereof, at a concentration of about 1 μM to about 100 mM, and optionally
(iv) further components, e.g. excipients, carriers or other active agents, e.g. as defined herein.

The instructions of component (iii) may for example comprise instructions to combine components (i) and (ii) (and (iv) if present) in sufficient amounts to prepare said antimicrobial aqueous composition or to combine components (i) and (ii) (and (iv) if present) with an aqueous diluent in sufficient amounts to prepare said antimicrobial aqueous composition.

In a further aspect the invention provides a kit comprising:
(i) a concentrated form of a ready to use antimicrobial aqueous composition described herein, or
(ii) a dry solid composition as described herein, and
(iii) instructions to prepare the ready to use form of the antimicrobial aqueous composition as described herein, e.g. by a method recited below.

In a still further aspect the invention provides a method of preparing a ready to use form of an antimicrobial aqueous composition as described herein, said method comprising:
(i) diluting a concentrated form of the ready to use composition with a sufficient amount of an aqueous diluent to form said ready to use antimicrobial aqueous composition, or
(ii) dissolving a dry solid composition as described herein in with a sufficient amount of an aqueous liquid to form said ready to use antimicrobial aqueous composition.

The above method may further comprise a step in which the ready to use antimicrobial aqueous composition is used in a method of combating contamination of a site with a microorganism, e.g. those methods described in detail above.

The dilution of the concentrated form of the antimicrobial aqueous composition and the dissolution of the dry solid composition may be achieved in a single step or a plurality of steps. The compositions may be added to the aqueous diluent/liquid, the aqueous diluent/liquid may be added to the compositions or each element may be combined simultaneously. In embodiments where a plurality of steps are employed, these may be additive/cumulative (repeated addition of aqueous diluent/liquid to the compositions, or portion thereof) or serial (repeated addition of the mixture of composition and aqueous diluent/liquid, or portion thereof, to more diluent/liquid) or a combination thereof. Preferably dilution is achieved in less than 10 steps, e.g. less than 9, 8, 7, 6, 5, 4, or 3 steps. More preferably dilution is achieved in 2 steps or, most conveniently, a single step.

In these latter two aspects the ready to use antimicrobial aqueous composition is generally defined as an antimicrobial aqueous composition comprising a decylglucoside at a concentration of about 2 mM to about 40 mM and at least one C₁ to C₈ carboxylic acid, or water-soluble salt thereof, at a concentration of about 1 μM to about 100 mM. In these latter two aspects the concentrated form of a ready to use antimicrobial aqueous composition described herein may be an antimicrobial aqueous composition consisting substantially, e.g. essentially, of
(i) a decylglucoside at a concentration of about 2 mM or greater,
(ii) at least one C₁ to C₈ carboxylic acid, or a water-soluble salt thereof, at a concentration of about 1 μM or greater, optionally
(iii) an octylglucoside at a concentration of about 0.2 mM (about 30 ppm) or greater, preferably wherein the ratio of said octylglucoside to said decylglucoside is at least about 10:100, optionally
(iv) an surface adherence agent at a concentration of about 50 μM or greater, and
(v) water.

In a further aspect the invention provides products susceptible to microbial contamination/colonisation whose susceptible surfaces have been pretreated with the antimicrobial aqueous compositions of the invention as defined herein.

By “pretreated” it is meant that the susceptible surface is exposed to an antimicrobial aqueous composition of the invention prior to exposure (or a further exposure) to a microorganism and that the antimicrobial components of the aqueous composition of the invention persist on the surface for a duration sufficient to prevent contamination/colonisation by a microorganism for an appreciable amount of time. In certain embodiments the antimicrobial components of the aqueous composition of the invention will persist for substantially the useful life of the surface, e.g. the pretreatment results in a substantially permanent coating of the antimicrobial components of the aqueous composition of the invention. Thus a pre-treated surface/product is one to which the antimicrobial components of the aqueous composition of the invention are applied and on which they remain. Such a product/surface may be a coated product/surface. Persistence/retention of the antimicrobial components may be accomplished by surface adherence and/or retention agents, e.g. PPL, present in the aqueous composition of the invention.

Non-limiting examples of products and surfaces susceptible to microbial contamination/colonisation are described above. Particular mention may be made of medical and surgical devices, food or drink processing, packaging, storage or dispensing equipment, in particular such equipment in meat processing plants and abattoirs and fruit and vegetable processing plants, and food and drink stuffs, in particular meats, vegetables and fruits and processed products containing the same.

Pretreatment can be achieved by any convenient means, for example any form of applying the aqueous composition of the invention to the surface, notably coating (e.g. spray coating) the surface.

“% w/v” (or “percentage weight by volume”) is a commonly used expression of the concentration of a solid solute in a liquid or semi-solid solution. 1% w/v equates to 1 gram of solid per 100 ml of solvent, 2% w/v equates to 2 g of solid per 100 ml of solvent, and so on. Accordingly local concentration may be expressed as g/100 ml, grams per 100 millilitres, g100 ml⁻¹. Likewise, “% w/w” (or “percentage weight by weight”) is a commonly used expression of the amount of a compound in a solid. 1% w/w equates to 1 gram of compound per 100 g of solid, 2% w/w equates to 2 g of compound per 100 g of solid, and so on. Accordingly, % w/w may be expressed as g/100 g, grams per 100 grams and g 100 g⁻¹. 1% w/w also equates to 10 gram of compound per kilogram of solid. Similarly, “parts-per-million” (ppm) is an expression of mg of solute per litre of solution or mg of compound per kg of solid. The skilled man would understand that through appropriate scaling calculations, the local concentration range of the present range can be expressed in terms of any SI unit of mass and volume.

The term “critical micelle concentration” refers to the concentration of a surfactant in a polar solvent at or above which the surfactant molecules will spontaneously arrange as a micelle. In the present context a micelle is considered to be a colloid arrangement of surfactant molecules in a polar solvent in which the hydrophilic head groups are oriented towards the polar solvent in a substantially spherical shape and the hydrophobic tail groups are sequestered to the interior of the sphere. In the present context a reference to the CMC of a surfactant is a reference to the CMC of the surfactant in the conditions of the aqueous composition of the invention (including physical conditions such as temperature), unless otherwise indicated. For convenience, this may be taken as the CMC of the surfactant in isolation in pure water at 20° C. CMC values may be calculated by any convenient means, preferably by one of the methods described in Mukerjee P., et. al, Critical Micelle Concentrations of Aqueous Surfactant Systems, United States Department of Commerce, Nat. Stnd. Ref. Data Ser., Nat. Bur. Stand., February 1971 (U.S.).

The invention will be further described with reference to the following non-limiting Examples.

### EXAMPLES

### Example 1

### Materials and Standard Methods:

APG: AG6210 (Akzo Nobel Surface Chemistry AB) 60% aqueous solution of a 60:40 mixture of decyl-β-D-glucopyranoside and octyl-β-D-glucopyranoside
   Decyl-β-D-glucopyranoside—EC number: 259-218-1; CAS number: 54549-25-6
   Octyl-β-D-glucopyranoside—EC number: 414-420-0; CAS number: 108081-06-7
ε-polylysine: Zhengzhou Bainafo Co., Ltd. CAS number: 28211-04-3
Lactic acid: L(+)-lactic acid. CAS number: 79-33-4
Citric acid: Citric acid. CAS number: 79-33-4
Neutraliser (NF): 30 ml Tween 80, 30 g saponine, 1 g histidine, 1 g cysteine dissolved in 1000 ml water; sterilised by autoclaving.
RBT 24/7: Residual Barrier Technology. Proprietary antimicrobial surface sanitizer.
Culture media: Oxoid plate count agar (CM0463):

| | g/litre |
|---|---|
| Yeast extract | 2.5 |
| Pancreatic digest of casein | 5.0 |
| Glucose | 10.0 |
| Agar | 15.0 |

pH 7.0±0.2 @ 25° C.

### Assay for Antimicrobial Activity:

Take 1 mL of test solutions and incubate with 10⁶ CFUs bacteria at room temperature for the specified time
Stop antimicrobial reaction by diluting with 10 mL of neutralizer
Plate 500 μL of neutralised bacterial reaction mixture (undiluted, 1:10, 1:100 and/or 1:1000 diluted) onto agar plates in triplicate
Incubate the inoculated plates at 37° C. for 24 hours
Assess the presence or absence of the microbial growth (+ plates with microbial growth and − for plates that had no growth).

### Example 2

### Antimicrobial Effects of Increasing Concentrations of L(+)-Lactic Acid

TABLE 1
Growth of E. coli 24 hrs after treatment with increasing concentrations of
lactic acid at RT for 30 minutes.

| Treatment/Control: | 1:10 dilution | | |
|---|---|---|---|
| mM L(+)-Lactic | of bacterial | 1:100 dilution of | 1:1000 dilution of |
| Acid + 10⁶ CFUs | reaction | bacterial reaction | bacterial reaction |
| 30 min RT | mixture | mixture | mixture |
| 50 mM | +++ | ++ | ++ |
| 100 mM | ++ | + | + |
| 250 mM | + | + | − |
| 500 mM | + | + | +/− |
| 1M | + | + | − |
| 2M | − | − | − |
| H₂O Control | +++ | +++ | +++ |
| Positive Control | − | − | − |
| RBT 24/7 1:30 | | | |

These results show that lactic acid can inhibit growth of E. coli after a 30 min treatment when used at at least 1M

### Example 3

### Antimicrobial Effects of Increasing Concentrations of Citric Acid

TABLE 2
Growth of E. coli 24 hrs after treatment with increasing concentrations of
citric acid at RT for 30 minutes.

| Treatment/ | 1:10 dilution of | | |
|---|---|---|---|
| Control: mM | bacterial | 1:100 dilution of | 1:1000 dilution of |
| Citric Acid + 10⁶ | reaction | bacterial reaction | bacterial reaction |
| CFUs 30 min RT | mixture | mixture | mixture |
| 1 mM | +++ | +++ | +++ |
| 2 mM | +++ | +++ | +++ |
| 3 mM | +++ | +++ | +++ |
| 5 mM | +++ | +++ | +++ |
| 7 mM | +++ | +++ | +++ |
| 10 mM | +++ | +++ | +++ |
| 25 mM | +++ | +++ | +++ |
| 50 mM | ++ | ++ | + |
| H₂O Control | +++ | +++ | +++ |
| Positive Control | − | − | − |
| RBT 24/7 1:30 | | | |

These results show that citric acid cannot inhibit growth of E. coli after a 30 min treatment when used at up to 50 mM

### Example 4

### Antimicrobial Effects of Increasing Concentrations of L(+)-Lactic Acid and Citric Acid Against E. Coli, Pseudomonas aeruginosa and Staphylococcus aureus

TABLE 3
Growth of E. coli 24 hrs after treatment with increasing concentrations of
L(+)-lactic acid and citric acid at RT for 30 minutes.

| Treatment/Control: | No dilution of | 1:10 dilution | 1:100 |
|---|---|---|---|
| mM L(+)-Lactic Acid + | bacterial | of bacterial | dilution of |
| mM Citric Acid + 10⁶ CFUs | reaction | reaction | bacterial |
| E. coli 30 min RT | mixture | mixture | reaction |
| 0.1 mM L(+)-Lactic Acid + | +++ | +++ | +++ |
| 0.1 mM Citric Acid | | | |
| 0.25 mM L(+)-Lactic Acid + | +++ | +++ | ++ |
| 0.25 mM Citric Acid | | | |
| 0.5 mM L(+)-Lactic Acid + | +++ | +++ | +++ |
| 0.5 mM Citric Acid | | | |
| 1 mM L(+)-Lactic Acid + | +++ | ++ | ++ |
| 1 mM Citric Acid | | | |
| 2 mM L(+)-Lactic Acid 2 mM | +++ | ++ | ++ |
| L(+)-Citric Acid | | | |
| 4 mM L(+)-Lactic Acid 4 mM | +++ | ++ | ++ |
| L(+)-Citric Acid | | | |
| H₂O Control | +++ | +++ | +++ |
| Positive Control RBT 24/7 | − | − | − |
| 1:30 | | | |

TABLE 4
Growth of Pseudomonas aeruginosa 24 hrs after treatment with increasing
concentrations of L(+)-lactic acid and citric acid at RT for 30 minutes.

| Treatment/Control: | | | 1:100 |
|---|---|---|---|
| mM L(+)-Lactic Acid + | No dilution | 1:10 dilution | dilution |
| mM Citric Acid + 10⁶ CFUs | of bacterial | of bacterial | of bacterial |
| Pseudomonas | reaction | reaction | reaction |
| aeruginosa 30 min RT | mixture | mixture | mixture |
| 0.1 mM L(+)-Lactic Acid + | +++ | +++ | +++ |
| 0.1 mM Citric Acid | | | |
| 0.25 mM L(+)-Lactic Acid + | +++ | +++ | ++ |
| 0.25 mM Citric Acid | | | |
| 0.5 mM L(+)-Lactic Acid + | +++ | +++ | +++ |
| 0.5 mM Citric Acid | | | |
| 1 mM L(+)-Lactic Acid + | +++ | ++ | ++ |
| 1 mM Citric Acid | | | |
| 2 mM L(+)-Lactic Acid + | +++ | +++ | ++ |
| 2 mM Citric Acid | | | |
| 4 mM L(+)-Lactic Acid + | +++ | ++ | ++ |
| 4 mM Citric Acid | | | |
| H₂O Control | +++ | +++ | +++ |
| Positive Control RBT 24/7 | − | − | − |
| 1:30 | | | |

TABLE 5
Growth of Staphylococcus aureus 24 hrs after treatment with increasing
concentrations of L(+)-lactic acid and citric acid at RT for 30 minutes.

| Treatment/Control: | | | 1:100 |
|---|---|---|---|
| mM L(+)-Lactic Acid + | No dilution | 1:10 dilution | dilution |
| mM Citric Acid + 10⁶ CFUs | of bacterial | of bacterial | of bacterial |
| Staphylococcus aureus | reaction | reaction | reaction |
| 30 min RT | mixture | mixture | mixture |
| 0.1 mM L(+)-Lactic Acid + | +++ | +++ | ++ |
| 0.1 mM Citric Acid | | | |
| 0.25 mM L(+)-Lactic Acid + | +++ | +++ | ++ |
| 0.25 mM Citric Acid | | | |
| 0.5 mM L(+)-Lactic Acid + | +++ | +++ | ++ |
| 0.5 mM Citric Acid | | | |
| 1 mM L(+)-Lactic Acid + | +++ | ++ | ++ |
| 1 mM Citric Acid | | | |
| 2 mM L(+)-Lactic Acid + | +++ | ++ | ++ |
| 2 mM Citric Acid | | | |
| 4 mM L(+)-Lactic Acid + | ++ | ++ | + |
| 4 mM Citric Acid | | | |
| H₂O Control | +++ | +++ | +++ |
| Positive Control RBT 24/7 | − | − | − |
| 1:30 | | | |

These results show that lactic acid and citric acid together cannot inhibit growth of E. coli, Pseudomonas aeruginosa or Staphylococcus aureus after a 30 min treatment when used at up to a combined concentration of 8 mM

### Example 5

### Antimicrobial Effects of Increasing Concentrations of APG Against E. Coli

TABLE 6
Growth of E. coli 24 hrs after treatment with increasing concentrations of
APG at RT for 30 minutes.

| | 1:10 dilution | 1:100 dilution | 1:1000 dilution |
|---|---|---|---|
| | of bacterial | of bacterial | of bacterial |
| x ppm APG + 10⁶ CFUs | reaction | reaction | reaction |
| E. coli 30 min RT | mixture | mixture | mixture |
| APG 1000 ppm | +++ | +++ | + |
| APG 10000 ppm | − | − | − |
| H₂O Control | +++ | +++ | +++ |
| Positive Control | − | − | − |
| RBT 24/7 1:30 | | | |

These results show that APG can inhibit growth of E. coli after a 30 min treatment when used at 10000 ppm but not 1000 ppm

### Example 6

### Antimicrobial Effects Against E. Coli of Increasing Concentrations of L(+)-Lactic Acid, Alone, in Combination With 1000 ppm APG, and in combination with 1000 ppm APG and 100 ppm ε-PPL

TABLE 7
Growth of E. coli 24 hrs after treatment with increasing concentrations of
L(+)-lactic acid, alone, in combination with 1000 ppm APG, and in
combination with 1000 ppm APG and
100 ppm ε-PPL at RT for 30 minutes.

| | L(+)- | L(+)- | L(+)-Lactic Acid + |
|---|---|---|---|
| Treatment/ | Lactic Acid | Lactic Acid + | 1000 ppm APG + |
| Control: | 10⁶ CFUs | 1000 ppm APG | 100 ppm PPL |
| mM L(+)- | E. coli | 10⁶ CFUs E. coli | 10⁶ CFUs E. coli |
| Lactic Acid | 30 min RT | 30 min RT | 30 min RT |
| 0.8 mM | +++ | − | − |
| 1.6 mM | +++ | − | − |
| 2.4 mM | +++ | − | − |
| 4 mM | +++ | − | − |
| 5.6 mM | +++ | − | − |
| 8 mM | +++ | − | − |
| 20 mM | +++ | +/− | − |
| 40 mM | +++ | − | − |
| H₂O Control | +++ | +++ | +++ |
| Positive Control | − | − | − |
| RBT 24/7 1:30 | | | |

TABLE 8
Growth of E. coli 24 hrs after treatment with increasing concentrations of
L(+)-lactic acid, alone, in combination with 1000 ppm APG,
and in combination with 1000 ppm APG and 100 ppm ε-PPL at RT
for 30 minutes (Repeat of Table 7 experiment).

| | L(+)- | L(+)- | L(+)-Lactic Acid + |
|---|---|---|---|
| Treatment/ | Lactic Acid | Lactic Acid + | 1000 ppm APG + |
| Control: | 10⁶ CFUs | 1000 ppm APG | 100 ppm PPL |
| mM L(+)- | E. coli | 10⁶ CFUs E. coli | 10⁶ CFUs E. coli |
| Lactic Acid | 30 min RT | 30 min RT | 30 min RT |
| 0.8 mM | +++ | − | − |
| 1.6 mM | +++ | − | − |
| 2.4 mM | +++ | − | − |
| 4 mM | +++ | − | − |
| 5.6 mM | +++ | − | − |
| 8 mM | +++ | − | − |
| 20 mM | +++ | +/− | − |
| 40 mM | +++ | − | − |
| H2O Control | +++ | +++ | +++ |
| Positive Control | − | − | − |
| RBT 24/7 1:30 | | | |

These results show that lactic acid alone cannot inhibit growth of E. coli after a 30 min treatment when used at up to 40 mM. However in the presence of 1000 ppm APG, or 1000 ppm APG and 100 ppm PPL, inhibition is observed with 0.8 mM lactic acid.

For ease of reference the following conversions for % concentrations of lactic acid are provided:
2%=220 mM
1%=110 mM
0.5%=55 mM
0.25%=27.5 mM
0.1%=11 mM
0.05%=5.5 mM
0.025%=2.75 mM
0.01%−1.1 mM

### Example 7

### Antimicrobial Effects of 5 mM L(+)-Lactic Acid and Increasing Concentrations of APG Against E. coli

TABLE 9
Growth of E. coli 24 hrs after treatment with 5 mM L(+)-lactic acid and
increasing concentrations of APG at RT for 30 minutes.

| | 1:10 | 1:100 | 1:1000 |
|---|---|---|---|
| | dilution of | dilution | dilution |
| 5 mM L(+)-Lactic Acid + | bacterial | of bacterial | of bacterial |
| x ppm APG + 10⁶ | reaction | reaction | reaction |
| CFUs E. coli 30 min RT | mixture | mixture | mixture |
| 100 | +++ | +++ | +++ |
| 250 | +++ | +++ | ++ |
| 500 | +++ | ++ | ++ |
| 750 | − | − | − |
| 1000 | − | − | − |
| 2500 | − | − | − |
| 5000 | − | − | − |
| 10000 | − | − | − |
| H₂O Control | +++ | +++ | +++ |
| Positive Control RBT 24/7 1:30 | − | − | − |

These results show that at least between 500 and 750 ppm APG are required for 5 mM lactic acid to inhibit the growth of E. coli upon a 30 min exposure. APG is a 60:40 mixture of decylglucoside and octylglucoside thus the effective reaction mixtures contain between 300 ppm and 450 ppm decylglucoside. The CMC of decylglucoside is approximately 300 ppm at 20° C.

### Example 8

### Time Dependence of the Antimicrobial Effects of 5 mM L(+)-Lactic Acid and 1000 ppm APG Against E. Coli

TABLE 10
Growth of E. coli 24 hrs after treatment with 5 mM L(+)-lactic acid
and 1000 ppm APG at RT for increasing time.

| | Contact time with 10⁶ CFUs E. coli at RT (mins) | | | | | | | |
|---|---|---|---|---|---|---|---|---|
| | 1 | 3 | 5 | 7.5 | 10 | 15 | 20 | 30 |
| 5 mM Lactic | − | − | − | − | − | − | − | − |
| acid + 1000 | | | | | | | | |
| ppm APG | | | | | | | | |
| 5 mM Lactic | − | − | − | − | − | − | − | − |
| acid + 1000 | | | | | | | | |
| ppm APG | | | | | | | | |
| 5 mM Lactic | − | − | − | − | − | − | − | − |
| acid + 1000 | | | | | | | | |
| ppm APG | | | | | | | | |
| Control (H₂O) | ++++ | ++++ | ++++ | ++++ | ++++ | ++++ | ++++ | ++++ |
| Control (H₂O) | ++++ | ++++ | ++++ | ++++ | ++++ | ++++ | ++++ | ++++ |
| Control (H₂O) | ++++ | ++++ | ++++ | ++++ | ++++ | ++++ | ++++ | ++++ |
| RBT 24/7 3% | − | − | − | − | − | − | − | − |
| RBT 24/7 3% | − | − | − | − | − | − | − | − |
| RBT 24/7 3%) | − | − | − | − | − | − | − | − |

These results shown that a solution consisting of 5 mM Lactic acid and 1000 ppm APG can inhibit growth of E. coli with exposure times of less than 1 minute.

### Example 9

### Antimicrobial Effects of Increasing Concentrations of Citric Acid in Combination With 1000 ppm APG

TABLE 11
Growth of E. coli 24 hrs after treatment with increasing concentrations of
citric acid in combination with 1000 ppm APG at RT for 30 minutes.

| Treatment/Control: | | | |
|---|---|---|---|
| mM Citric Acid + | 1:10 dilution | 1:100 dilution | 1:1000 dilution |
| 1000 ppm APG + | of bacterial | of bacterial | of bacterial |
| 10⁶ CFUs E. coli 30 min | reaction | reaction | reaction |
| RT | mixture | mixture | mixture |
| 1 mM | − | − | − |
| 2 mM | − | − | − |
| 3 mM | − | − | − |
| 5 mM | − | − | − |
| 7 mM | − | − | − |
| 10 mM | − | − | − |
| 25 mM | − | − | − |
| 50 mM | − | − | − |
| H₂O Control | +++ | +++ | +++ |
| Positive Control | − | − | − |
| RBT 24/7 1:30 | | | |

These results show that in the presence of 1000 ppm APG citric acid can inhibit the growth of E. coli at concentrations of at least 1 mM upon a 30 min exposure.

### Example 10

### Antimicrobial Effects of Increasing Concentrations of Citric Acid in Combination With 1000 ppm APG and 100 ppm ε-PPL

TABLE 12
Growth of E. coli 24 hrs after treatment with increasing concentrations of
citric acid in combination with 1000 ppm APG and 100 ppm ε-PPL
at RT for 30 minutes.

| Treatment/Control: | | | |
|---|---|---|---|
| mM Citric Acid + | 1:10 dilution | 1:100 dilution | 1:1000 dilution |
| 1000 ppm APG + | of bacterial | of bacterial | of bacterial |
| 100 ppm ε-PPL 10⁶ | reaction | reaction | reaction |
| CFUs E. coli 30 min RT | mixture | mixture | mixture |
| 1 mM | − | − | − |
| 2 mM | − | − | − |
| 3 mM | − | − | − |
| 5 mM | − | − | − |
| 7 mM | − | − | − |
| 10 mM | − | − | − |
| 25 mM | − | − | − |
| 50 mM | − | − | − |
| H₂O Control | +++ | +++ | +++ |
| Positive Control | − | − | − |
| RBT 24/7 1:30 | | | |

These results show that in the presence of 1000 ppm APG and 100 pm PPL, citric acid can inhibit the growth of E. coli at concentrations of at least 1 mM upon a 30 min exposure.

### Example 11

### Antimicrobial Effects Against E. coli of Increasing Concentrations of L(+)-Lactic Acid and Citric Acid, Alone, in Combination With 1000 ppm APG, and in Combination With 1000 ppm APG and 100 ppm ε-PPL

Tables 13 to 15: Growth of E. coli 24 hrs after treatment with increasing concentrations of L(+)-lactic acid and citric acid, alone, in combination with 1000 ppm APG, and in combination with 1000 ppm APG and 100 ppm ε-PPL at RT for 30 minutes.

TABLE 13

| | 1:10 dilution | 1:100 | 1:1000 | |
|---|---|---|---|---|
| Treatment/Control: | of bacterial | dilution of | dilution of | |
| mM L(+)-Lactic Acid + mM Citric Acid + | reaction | bacterial | bacterial | |
| 10⁶ CFUs E. coli 30 min RT | mixture | reaction | reaction | pH |
| 100 μM Lactic Acid + 100 μM Citric Acid | +++ | +++ | +++ | 3.5 |
| 250 μM Lactic Acid + 250 μM Citric Acid | +++ | +++ | +++ | 3.3 |
| 500 μM Lactic Acid + 500 μM Citric Acid | +++ | +++ | +++ | 3.18 |
| 1000 μM Lactic Acid + 1000 μM Citric Acid | +++ | +++ | +++ | 3.1 |
| 2 mM Lactic Acid + 2 mM Citric Acid | +++ | +++ | +++ | 2.9 |
| 4 mM Lactic Acid + 4 mM Citric Acid | +++ | +++ | +++ | 2.7 |
| H₂O Control | +++ | +++ | +++ | 8.24 |
| Positive Control RBT 24/7 1:30 | − | − | − | 7.65 |

TABLE 14

| Treatment/Control: | 1:10 dilution | 1:100 | 1:1000 | |
|---|---|---|---|---|
| mM L(+)-Lactic Acid + mM Citric Acid + | of bacterial | dilution of | dilution of | |
| 1000 ppm APG + 10⁶ CFUs E. coli 30 min | reaction | bacterial | bacterial | |
| RT | mixture | reaction | reaction | pH |
| 100 μM Lactic Acid + 100 μM Citric Acid | − | − | − | 8.72 |
| 250 μM Lactic Acid + 250 μM Citric Acid | − | − | − | 8.09 |
| 500 μM Lactic Acid + 500 μM Citric Acid | − | − | − | 8.07 |
| 1000 μM Lactic Acid + 1000 μM Citric Acid | − | − | − | 4.8 |
| 2 mM Lactic Acid + 2 mM Citric Acid | − | − | − | 3.6 |
| 4 mM Lactic Acid + 4 mM Citric Acid | − | − | − | 3.42 |
| H₂O Control | +++ | +++ | +++ | 8.24 |
| Positive Control RBT 24/7 1:30 | − | − | − | 7.65 |

TABLE 15

| Treatment/Control: | 1:10 dilution | 1:100 | 1:1000 | |
|---|---|---|---|---|
| mM Citric Acid + 1000 ppm APG + | of bacterial | dilution of | dilution of | |
| 100 ppm ε-PPL 10⁶ CFUs E. coli 30 min | reaction | bacterial | bacterial | |
| RT | mixture | reaction | reaction | pH |
| 100 μM Lactic Acid + 100 μM Citric Acid | − | − | − | 3.25 |
| 250 μM Lactic Acid + 250 μM Citric Acid | − | − | − | 3.19 |
| 500 μM Lactic Acid + 500 μM Citric Acid | − | − | − | 3.17 |
| 1000 μM Lactic Acid + 1000 μM Citric Acid | − | − | − | 2.86 |
| 2 mM Lactic Acid + 2 mM Citric Acid | − | − | − | 2.77 |
| 4 mM Lactic Acid + 4 mM Citric Acid | − | − | − | 2.63 |
| H₂O Control | +++ | +++ | +++ | 8.24 |
| Positive Control RBT 24/7 1:30 | − | − | − | 7.65 |

These results show that a combination of lactic acid and citric acid alone cannot inhibit growth of E. coli after a 30 min treatment when used at up to 8 mM. However in the presence of 1000 ppm APG, or 1000 ppm APG and 100 ppm PPL, inhibition is observed with at least 200 μM of the acids.

### Example 12

### Antimicrobial Effects Against E. coli of Increasing Concentrations of L(+)-Lactic Acid and Citric Acid, Alone, in Combination With 1000 ppm APG, and in Combination With 1000 ppm APG and 100 ppm ε-PPL at Approximately Neutral pH

Tables 16 to 18: Growth of E. coli 24 hrs after treatment with increasing concentrations of L(+)-lactic acid and citric acid, alone, in combination with 1000 ppm APG, and in combination with 1000 ppm APG and 100 ppm ε-PPL at RT and approximately neutral pH for 30 minutes.

TABLE 16

| | 1:10 | 1:100 | 1:1000 |
|---|---|---|---|
| Treatment/Control: | dilution | dilution of | dilution of |
| mM L(+)- | of bacterial | bacterial | bacterial |
| Lactic Acid + mM Citric Acid + | reaction | reaction | reaction |
| 10⁶ CFUs E. coli 30 min RT | mixture | mixture | mixture |
| 100 μM Lactic Acid + 100 μM | +++ | +++ | +++ |
| Citric Acid | | | |
| 250 μM Lactic Acid + 250 μM | +++ | +++ | +++ |
| Citric Acid | | | |
| 500 μM Lactic Acid + 500 μM | +++ | +++ | +++ |
| Citric Acid | | | |
| 1000 μM Lactic Acid + 1000 μM | +++ | +++ | +++ |
| Citric Acid | | | |
| 2 mM Lactic Acid + 2 mM | +++ | +++ | +++ |
| Citric Acid | | | |
| 4 mM Lactic + 4 mM Citric | +++ | +++ | +++ |
| Acid | | | |
| H₂O Control | +++ | +++ | +++ |
| Positive Control RBT 24/7 1:30 | − | − | − |

TABLE 17

| Treatment/Control: | 1:10 | | |
|---|---|---|---|
| mM L(+)- | dilution | 1:100 | 1:1000 |
| Lactic Acid + mM Citric Acid + | of bacterial | dilution of | dilution of |
| 1000 ppm APG + | reaction | bacterial | bacterial |
| 10⁶ CFUs E. coli 30 min RT | mixture | reaction | reaction |
| 100 μM Lactic Acid + 100 μM | − | − | − |
| Citric Acid | | | |
| 250 μM Lactic Acid + 250 μM | − | − | − |
| Citric Acid | | | |
| 500 μM Lactic Acid + 500 μM | − | − | − |
| Citric Acid | | | |
| 1000 μM Lactic Acid + 1000 μM | − | − | − |
| Citric Acid | | | |
| 2 mM Lactic Acid + 2 mM | − | − | − |
| Citric Acid | | | |
| 4 mM Lactic Acid + 4 mM | − | − | − |
| Citric Acid | | | |
| H₂O Control | +++ | +++ | +++ |
| Positive Control RBT 24/7 1:30 | − | − | − |

TABLE 18

| Treatment/Control: | 1:10 | 1:100 | 1:1000 |
|---|---|---|---|
| mM L(+)-Lactic | dilution of | dilution of | dilution of |
| Acid + mM Citric Acid + | bacterial | bacterial | bacterial |
| 1000 ppm APG + 10⁶ CFUs | reaction | reaction | reaction |
| E. coli 30 min RT | mixture | mixture | mixture |
| 50 μM Lactic + 50 μM Citric Acid | − | − | − |
| 125 μM Lactic + 125 μM Citric Acid | − | − | − |
| 250 μM Lactic + 250 μM Citric Acid | − | − | − |
| 500 μM Lactic + 500 μM Citric Acid | − | − | − |
| 1 mM Lactic + 1 mM Citric Acid | − | − | − |
| 2 mM Lactic + 2 mM Citric Acid | − | − | − |
| H₂O Control | +++ | +++ | +++ |
| Positive Control RBT 24/7 1:30 | − | − | − |

TABLE 19

| +Treatment/Control: | | 1:100 | 1:1000 |
|---|---|---|---|
| mM Citric Acid + | 1:10 dilution | dilution of | dilution of |
| 1000 ppm APG + 100 ppm | of bacterial | bacterial | bacterial |
| ε-PPL 10⁶ CFUs | reaction | reaction | reaction |
| E. coli 30 min RT | mixture | mixture | mixture |
| 100 μM Lactic Acid + 100 μM | − | − | − |
| Citric Acid | | | |
| 250 μM Lactic Acid + 250 μM | − | − | − |
| Citric Acid | | | |
| 500 μM Lactic Acid + 500 μM | − | − | − |
| Citric Acid | | | |
| 1000 μM Lactic Acid + 1000 μM | − | − | − |
| Citric | | | |
| 2 mM Lactic Acid + 2 mM | − | − | − |
| Citric Acid | | | |
| 4 mM Lactic Acid + 4 mM | − | − | − |
| Citric Acid | | | |
| H₂O Control | +++ | +++ | +++ |
| Positive Control RBT 24/7 1:30 | − | − | − |

These results show that a combination of lactic acid and citric acid alone cannot inhibit growth of E. coli after a 30 min treatment when used at up to 8 mM. However in the presence of 1000 ppm APG, or 1000 ppm APG and 100 ppm PPL, inhibition is observed with at least 200 μM of the acids. This effect is independent of an acidic pH.

### Example 13

### Time Dependence of the Antimicrobial Effects Against E. coli of Increasing Concentrations of L(+)-Lactic Acid and Citric Acid in Combination With 500 ppm or 1000 ppm APG

TABLE 20
Growth of E. coli 24 hrs after treatment with increasing concentrations of
L(+)-lactic acid and citric acid in combination with
500 ppm APG at RT for increasing time.

| Treatment/Control: | Contact time with | | |
|---|---|---|---|
| mM L(+)-Lactic Acid + mM Citric Acid + | 10⁶ CFUs E. coli at RT | | |
| 500 ppm APG | 1 min | 3 min | 5 min |
| 50 μM Lactic Acid + 50 μM Citric Acid | +++ | ++ | + |
| 100 μM Lactic Acid + 100 μM Citric Acid | ++ | + | − |
| 125 μM Lactic Acid + 125 μM Citric Acid | ++ | + | − |
| 1000 μM Lactic Acid + 1000 μM Citric Acid | ++ | + | − |
| 2.5 mM Lactic + 2.5 mM Citric Acid | − | − | − |
| H₂O Control | +++ | +++ | +++ |
| Positive Control RBT 24/7 1:30 | − | − | − |

TABLE 21
Growth of E. coli 24 hrs after treatment with increasing concentrations of
L(+)-lactic acid and citric acid in combination with
1000 ppm APG at RT for increasing time.

| Treatment/Control: | Contact time with | | |
|---|---|---|---|
| mM L(+)-Lactic Acid + mM Citric Acid + | 10⁶ CFUs E. coli at RT | | |
| 1000 ppm APG | 1 min | 3 min | 5 min |
| 50 μM Lactic + 50 μM Citric Acid | − | − | − |
| 100 μM Lactic + 100 μM Citric Acid | − | − | − |
| 125 μM Lactic + 125 μM Citric Acid | − | − | − |
| 1000 μM Lactic + 1000 μM Citric Acid | − | − | − |
| 2.5 mM Lactic + 2.5 mM Citric Acid | − | − | − |
| H₂O Control | +++ | +++ | +++ |
| Positive Control RBT 24/7 1:30 | − | − | − |

These results show that in the presence of 500 ppm APG only a combination of lactic/citric acid with a combined concentration of 5 mM alone can inhibit growth of E. coli after a 1 or 3 min. Longer incubation of 5 min has greater effects at lower acid concentrations. In contrast, in the presence of 1000 ppm APG inhibition is observed with at least 100 μM lactic acid/citric acid and within a 1min incubation.

### Example 14

### Antimicrobial Effects Against E. coli of Increasing Concentrations of L(+)-Lactic Acid and Citric Acid in Combination With Increasing Concentrations of APG

TABLE 22
Growth of E. coli 24 hrs after treatment with increasing concentrations of
L(+)-lactic acid and citric acid in combination with
increasing concentrations of APG at RT for 30 min

| | Combined concentration of L(+)- | | | | |
|---|---|---|---|---|---|
| Treatment/Control: | Lactic Acid + mM Citric Acid (μM) | | | | |
| ppm APG 10⁶ CFUs 3 min RT | 5 | 10 | 25 | 100 | 250 |
| 100 | +++ | +++ | +++ | +++ | +++ |
| 250 | +++ | +++ | +++ | +++ | +++ |
| 300 | +++ | +++ | +++ | +++ | +++ |
| 400 | ++ | ++ | ++ | ++ | ++ |
| 500 | + | + | + | + | + |
| 750 | − | − | − | − | − |
| H₂O Control | +++ | +++ | +++ | +++ | +++ |
| Positive Control RBT 24/7 1:30 | − | − | − | − | − |

These results show that at least between 500 and 750 ppm APG are required for a 5 μM to 250 μM lactic and citric acid combination to inhibit the growth of E. coli upon a 3 min exposure. APG is a 60:40 mixture of decylglucoside and octylglucoside thus the effective reaction mixtures contain between 300 ppm and 450 ppm decylglucoside. The CMC of decylglucoside is approximately 300 ppm at 20° C.

1. A method for combating contamination of a site with a microorganism, said method comprising contacting the site and/or the microorganism with an antimicrobial aqueous composition comprising:
(i) a decylglucoside at a concentration of about 2 mM to about 40 mM, and
(ii) at least one C₁ to C₈ carboxylic acid, or a water-soluble salt thereof, at a concentration of about 1 μM to about 100 mM.

2. The method of claim 1, wherein the decylglucoside is decyl β-D-glucopyranoside.

3. The method of claim 1 or claim 2, wherein the C₁ to C₈ carboxylic acid is an alpha hydroxy acid, preferably selected from lactic acid, citric acid, glycolic acid, malic acid, mandelic acid and tartaric acid.

4. The method of any one of claims 1 to 3, wherein the C₁ to C₈ carboxylic acid is a C₃ to C₆ carboxylic acid.

5. The method of claim 1 or claim 2 wherein the C₁ to C₈ carboxylic acid is selected from lactic acid, citric acid, glycolic acid, malic acid, mandelic acid, tartaric acid, butyric acid, succinic acid, sorbic acid, isocitric acid, aconitic acid, propane-1,2,3-tricarboxylic acid, propionic acid, valeric acid, benzoic acid, oxalic acid or salicylic acid, preferably lactic acid and/or citric acid.

6. The method of any one of claims 1 to 5, wherein the concentration of the C₁ to C₈ carboxylic acid is equal to or less than about 10 mM

7. The method of claim 6, wherein the concentration of the C₁ to C₈ carboxylic acid is equal to or less than about 2.5 mM.

8. The method of any one of claims 1 to 7, wherein the composition consists of water and the decylglucoside and the at least one C₁ to C₈ carboxylic acid dissolved therein.

9. The method of any one of claims 1 to 7, wherein the composition further comprises an octylglucoside at a concentration of about 0.2 mM to about 200 mM, preferably at a ratio of the octylglucoside to the decylglucoside of at least about 10:100, preferably about 40:60.

10. The method of claim 9, wherein the octylglucoside is octyl β-D-glucopyranoside.

11. The method of claim 9 or claim 10, wherein the composition consists of water and the decylglucoside, the octylglucoside and the at least one C₁ to C₈ carboxylic acid dissolved therein.

12. The method of any one of claim 1 to 7, 9 or 10, wherein the composition further comprises an agent to enhance the surface adherence of the antimicrobial components at a concentration of about 50 μM to about 10 mM.

13. The method of claim 12, wherein said agent to enhance the surface adherence of the antimicrobial components is selected from ε polylysine (PPL), abaecin, bactenecin, lactoferricin B, cecropin C, preferably PPL.

14. The method of claim 12 or claim 13 wherein the composition consists of water and the decylglucoside, the octylglucoside, the at least one C₁ to C₈ carboxylic acid and the agent to enhance the surface adherence of the antimicrobial components dissolved therein.

15. The method of any one of claims 1 to 14, wherein the composition is liquid.

16. The method of any one of claim 1 to 7, 9, 10, 12 or 13 wherein the composition further comprises carriers that influence the viscosity and/or rheology of the composition.

17. The method of claim 16, wherein the composition is a viscous liquid, a semi solid or a solid, preferably a hydrogel or hydrocolloid gel.

18. The method of any one of claims 1 to 17, wherein said microorganism is on an inanimate surface.

19. The method of claim 18, wherein the site is a surface selected from surfaces of food or drink processing, preparation, storage, packaging or dispensing machinery or equipment, abattoir machinery or equipment, fruit and vegetable processing or packaging machinery or equipment; surfaces of air conditioning apparatus; surfaces of industrial machinery, surfaces of storage tanks; surfaces of medical or surgical equipment; surfaces of clinical beds, furniture, floors, doors, door handles, operating theatres, office and IT equipment; cell and tissue culture equipment; surfaces of sanitaryware and fittings; surfaces of aquatic/marine equipment, the surfaces of buildings and other structures or the surfaces of food, preferably meat, fish, vegetables and fruit, and food packaging.

20. The method of claim 18, wherein the site is in or on an internal or external body surface.

21. The method of claim 20 wherein the internal or external body surface is selected from a surface in the oral cavity, the reproductive tract, the urinary tract, the respiratory tract, the gastrointestinal tract, the peritoneum, the middle ear, the prostate, vascular intima, the eye, including conjunctiva or corneal tissue, lung tissue, heart valves, skin, scalp, nails, the interior of wounds or the surface of adrenal, hepatic, renal, pancreatic, pituitary, thyroid, immune, ovarian, testicular, prostate, endometrial, ocular, mammary, adipose, epithelial, endothelial, neural, muscle, pulmonary, epidermis or osseous tissue.

22. An antimicrobial aqueous composition as defined in any one of claims 1 to 17 for use as a therapeutic microbicidal and/or microbiostatic agent.

23. An antimicrobial aqueous composition as defined in any one of claims 1 to 17 for use in inhibiting the viability and/or growth of a microorganism in or on a subject.

24. An antimicrobial aqueous composition as defined in any one of claims 1 to 17 for use in combating microbial infection in or on a subject.

25. The antimicrobial aqueous composition for the use of either claim 23 or claim 24, wherein the microorganism or the microbial infection is in or on an internal or external body surface.

26. The antimicrobial aqueous composition for the use of claim 25 wherein the internal or external body surface is selected from a surface in the oral cavity, the reproductive tract, the urinary tract, the respiratory tract, the gastrointestinal tract, the peritoneum, the middle ear, the prostate, vascular intima, the eye, including conjunctiva or corneal tissue, lung tissue, heart valves, skin, scalp, nails, the interior of wounds or the surface of adrenal, hepatic, renal, pancreatic, pituitary, thyroid, immune, ovarian, testicular, prostate, endometrial, ocular, mammary, adipose, epithelial, endothelial, neural, muscle, pulmonary, epidermis or osseous tissue.

27. An antimicrobial aqueous composition, wherein said composition is as defined in any one of claims 1 to 17.

28. An antimicrobial aqueous composition consisting substantially of
(i) a decylglucoside at a concentration of about 2 mM or greater,
(ii) at least one C₁ to C₈ carboxylic acid, or a water-soluble salt thereof, at a concentration of about 1 μM or greater, optionally
(iii) an octylglucoside at a concentration of about 0.2 mM or greater, preferably wherein the ratio of said octylglucoside to said decylglucoside is at least about 10:100, optionally
(iv) a surface adherence agent at a concentration of about 50 μM or greater, and
(v) water

29. A dry solid composition comprising amounts of a decylglucoside and at least one C₁ to C₈ carboxylic acid, or a water-soluble salt thereof, that following contact with a sufficient amount of an aqueous liquid provides a ready to use form of the antimicrobial aqueous composition as defined in any one of claims 1 to 17.

30. A kit comprising, optionally in separate containers:
(i) a decylglucoside, optionally in an aqueous solution
(ii) at least one C₁ to C₈ carboxylic acid, or a water-soluble salt thereof, optionally in an aqueous solution; and
(iii) instructions to prepare an antimicrobial aqueous composition as defined in any one of claims 1 to 17, and optionally
(iv) further components, e.g. excipients, carriers or other active agents.

31. A kit comprising:
(i) a concentrated form of the antimicrobial aqueous composition as defined in any one of claims 1 to 17, or
(ii) a dry solid composition of the invention as defined in claim 29, and
(iii) instructions to prepare a ready to use form of the antimicrobial aqueous composition as defined in any one of claims 1 to 17.

32. A method of preparing a ready to use antimicrobial aqueous composition as defined in any one of claims 1 to 17, said method comprising:
(i) diluting a concentrated form of the composition as defined in any one of claims 1 to 17 with a sufficient amount of an aqueous diluent to form said ready to use antimicrobial aqueous composition, or
(ii) dissolving a dry solid composition as defined in claim 29 with a sufficient amount of an aqueous liquid to form said ready to use antimicrobial aqueous composition.

33. The method of claim 32 further comprising a step in which the ready to use antimicrobial aqueous composition is used in a method of combating contamination of a site with a microorganism as defined is any one of claims 1 to 17.

34. A product susceptible to microbial contamination comprising a surface which has been pretreated with the antimicrobial aqueous composition as defined in any one of claims 1 to 17.
